(19) United States
(12) Patent Application Publication
(10) Pub. No.: US 2026/0229315 A1
(43) Pub. Date: Aug. 6, 2026

(54) COMPUTER-IMPLEMENTED MACHINE LEARNING SYSTEM FOR MYCOBACTERIUM BOVIS DETECTION USING MICRORNA BIOMARKER CLASSIFICATION

(71) Applicant: MI:RNA LTD., EDINBURGH (GB)

(72) Inventors: Eve HANKS, EDINBURGH (GB); Robert COULTOUS, EDINBURGH (GB)

(73) Assignee: MI:RNA LTD., EDINBURGH (GB)

(21) Appl. No.: 19/532,120

(22) Filed: Feb. 6, 2026

Related U.S. Application Data
(60) Provisional application No. 63/754,988, filed on Feb. 6, 2025.

(51) Int. Cl. G16B 40/20; G06N 20/20; G16B 25/10

(52) U.S. Cl. CPC G16B 40/20; G06N 20/20; G16B 25/10

(57) ABSTRACT

A computer-implemented ensemble machine learning system for detecting Mycobacterium bovis infection processes qPCR-derived miRNA expression data through trained classification algorithms. The system receives quantitative data for a miRNA panel, calculates delta-Cq values, in some embodiments normalized said delta-Cq values, and applies classification models. The machine learning engine generates classification probability scores processed through threshold comparison logic to produce binary diagnostic classifications with 91% accuracy, 0.89 sensitivity, and 0.92 specificity. In some embodiment, an ensemble approach computationally combines multiple algorithmic outputs to improve diagnostic reliability over single-classifier systems, enabling automated bovine tuberculosis detection that overcomes technical limitations of conventional testing methods for rapid, field-deployable herd management decisions.

### CROSS-REFERENCE TO RELATED APPLICATIONS

This application claims the benefit of priority to U.S. Provisional Application No. 63/754,988, filed Feb. 6, 2025, the entire contents of which are incorporated herein by reference.

### REFERENCE TO ELECTRONIC SEQUENCE LISTING

The application contains a Sequence Listing which has been submitted electronically in .XML format and is hereby incorporated by reference in its entirety. Said. XML copy, created on, is named “068075.014US.xml” and is bytes in size. The sequence listing contained in this .XML file is part of the specification and is hereby incorporated by reference herein in its entirety.

### TECHNICAL FIELD

This invention relates generally to isolated nucleic acid molecules known as microRNAs (miRNAs) and miRNA precursor molecules and their use in diagnosis and therapy. The invention also relates to a method and a kit for diagnosing, monitoring, and treating bovine tuberculosis.

### BACKGROUND

Bovine tuberculosis (bTB), caused primarily by Mycobacterium bovis, imposes substantial animal-health and economic burdens through chronic respiratory disease, reduced growth and milk yield, premature mortality, and compulsory culling of infected or exposed animals. In the United Kingdom bTB is a notifiable disease subject to statutory surveillance, movement restrictions, and eradication measures. Current statutory and supplementary diagnostics—principally the Single Intradermal Comparative Cervical Tuberculin (SICCT) skin test and the interferon-gamma blood assay—have imperfect sensitivity and specificity, are influenced by host immune status and environmental mycobacteria, and often require sequential or confirmatory testing (culture or molecular assays) that delays definitive results. These diagnostic limitations reduce the ability to detect early or latent infections, permit undetected within-herd transmission, prolong movement restrictions, and increase economic and operational burdens on producers and veterinary services.

There is a need for a rapid, accurate, and field-deployable diagnostic that reliably detects early and latent M. bovis infection, minimizes unnecessary culling, and supports timely herd-level control measures. The present invention addresses this need by using quantitative measurement of a defined panel of bovine microRNAs (miRNAs), measured by qPCR, in combination with a trained statistical or machine-learning classifier to generate a diagnostic output indicative of bTB infection status.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a computer-implemented method for detecting Mycobacterium bovis infection in a subject. The computer-implemented method also includes receiving, by a processor, quantitative expression data for a panel of microRNA (miRNA) molecules from a biological sample obtained from the subject. In some embodiments, the method also includes normalizing, by the processor, the quantitative expression data by calculating a delta quantification cycle (delta Cq) value for each target miRNA in the panel relative to a mean quantification cycle (Cq) value of one or more endogenous control miRNA. The method also includes applying, by the processor, a trained classification model to the normalized or non-normalized expression data, where the trained classification model is configured to classify the expression data as control or reactor. The method also includes generating, by the processor, a classification output indicative of Mycobacterium bovis infection status of the subject based on the applying. Other embodiments of this aspect include corresponding computer systems, apparatus, and computer programs recorded on one or more computer storage devices, each configured to perform the actions of the methods.

Implementations may include one or more of the following features. The method where the panel of miRNA molecules may include mir-128, mir-17, mir-16, mir-21, and mir-423. The one or more endogenous control miRNA may include mir-21 and mir-423, and the delta Cq value for each target miRNA is calculated relative to the mean Cq value of mir-21 and mir-42. The trained classification model may include a discriminant analysis classifier. The discriminant analysis classifier may include a quadratic discriminant analysis classifier, a partial least squares classifier, a random forest classifier, or a support vector machine (SVM) classifier. The trained classification model is trained on a reference dataset may include expression profiles from control samples and reactor samples using cross-validated training. The cross-validated training may include ten-fold cross-validation repeated five times. The biological sample is selected from the group may include of serum, plasma, and whole blood. The method detects early infection or latent infection with Mycobacterium bovis. Implementations of the described techniques may include hardware, a method or process, or computer software on a computer-accessible medium.

One general aspect includes a system for detecting Mycobacterium bovis infection. The system also includes a memory storing a trained classification model configured to classify microRNA (miRNA) expression data as control or reactor, and instructions. The system also includes a processor coupled to the memory, where execution of the instructions by the processor causes the processor to: receive quantitative expression data for a panel of miRNA molecules from a biological sample, normalize the quantitative expression data, apply the trained classification model to the normalized expression data, and generate a classification output indicative of Mycobacterium bovis infection status. In some embodiments, the method includes normalizing the delta Cq values by one or more techniques selected from normalizing the delta Cq values relative to a mean quantification cycle (Cq) value of one or more endogenous control miRNAs, global mean normalization, independent standardization, pairwise normalization, or a combination thereof. Other embodiments of this aspect include corresponding computer systems, apparatus, and computer programs recorded on one or more computer storage devices, each configured to perform the actions of the methods.

Implementations may include one or more of the following features. The system where the panel of miRNA molecules may include mir-128, mir-17, mir-16, mir-21, and mir-423. The one or more endogenous control miRNAs may include mir-21 and mir-423, and the delta Cq value for each target miRNA is calculated relative to the mean Cq value of mir-21 and mir-423. The trained classification model may include a discriminant analysis classifier. The discriminant analysis classifier may include a quadratic discriminant analysis classifier, a partial least squares classifier, a random forest classifier, or a support vector machines (SVM) classifier. The trained classification model is trained on a reference dataset may include expression profiles from control samples and reactor samples using cross-validated training. The cross-validated training may include ten-fold cross-validation repeated five times. The panel of miRNA molecules may include mir-17, mir-16, mir-22, mir-128, mir-148a, mir-320a, mir-320b, mir-21, mir-423, and mir-193a. The system is configured to detect early infection or latent infection with Mycobacterium bovis. Implementations of the described techniques may include hardware, a method or process, or computer software on a computer-accessible medium.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

FIGS. 1A-B depict the differences observed between control and reactor samples. FIG. 1A is a PCA plot showing multivariate structure separating control and reactor samples. FIG. 1B shows a hierarchical clustering heatmap of retained miRNA features, showing distinct expression patterns between groups.

FIGS. 2A-D show the accuracy metrics of various machine learning-trained models.

FIG. 3 is a ten-fold cross-validated receiver operating curve (ROC) for the QDA model. Area under the curve (AUC) is shown.

FIGS. 4A-B depict the differences between control and reactor samples in a minimal panel. FIG. 4A is a PCA plot showing multivariate separation of control and reactor samples. FIG. 4B is a heatmap showing distinct separation per group.

FIG. 5 is a ten-fold cross-validated receiver operating curve (ROC) for the PLS model. Area under the curve (AUC) is shown.

FIGS. 6A-D show the accuracy metrics of various machine learning-trained models.

FIG. 7 is a block diagram showing the diagnostic system architecture for detecting Mycobacterium bovis infection.

FIG. 8 is a flowchart illustrating the computer-implemented method steps from sample collection through diagnostic report output.

FIGS. 9A-B are flowcharts depicting the machine learning model training process using control and reactor samples. FIG. 9B depicts a specific embodiment of said training process.

FIG. 10A-B is a flowchart showing the classification workflow for processing new miRNA expression data through trained models. FIG. 10B depicts a specific embodiment of said workflow.

FIG. 11 is a block diagram illustrating the detailed computer system architecture with hardware and software components.

FIG. 12 is a flowchart showing the complete diagnostic workflow from subject evaluation through herd management decisions.

FIG. 13 is a cross-validated receiver operating curve (ROC) for the random forest model. Area under the curve (AUC) is shown.

FIG. 14 is a cross-validated receiver operating curve (ROC) for the support vector machines (SVM) model, with global mean normalization and independent standardization. Area under the curve (AUC) is shown.

FIG. 15 is a cross-validated receiver operating curve (ROC) for the support vector machines (SVM) model, with pairwise normalization. Area under the curve (AUC) is shown.

### DETAILED DESCRIPTION OF THE INVENTION

The present invention may be understood more readily by reference to the following detailed description of preferred embodiments of the invention and the Examples included therein and to the Figures and their previous and following description.

### I Definitions

The following definitions are provided to facilitate understanding of certain terms used throughout this disclosure.

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The terms “embodiment,” “an embodiment,” “one embodiment,” “in various embodiments,” “certain embodiments,” “some embodiments,” “other embodiments,” “certain other embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with any other embodiment whether or not explicitly described.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

A “coding sequence” or “coding region” refers to a nucleic acid molecule having sequence information necessary to produce a gene product, when the sequence is expressed.

A “probe” is defined as a nucleic acid capable of binding to a target nucleic acid of complementary sequence through one or more types of chemical bonds, usually through complementary base pairing, usually through hydrogen bond formation. A probe may include natural (i.e., A, G, C, T or U) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. Probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

As used herein, the term “microRNA” or “miRNA” or “miR” designates a non-coding RNA molecule having a length of about 17 to 25 nucleotides, specifically having a length of 17, 18, 19, 20, 21, 22, 23, 24 or 25 nucleotides which hybridizes to and regulates the expression of a coding messenger RNA.

The term “miRNA molecule” refers to any nucleic acid molecule representing the miRNA, including natural miRNA molecules, i.e. the mature miRNA, pre-miRNA, pri-miRNA.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “sample” generally refers to tissue or organ sample, blood, cell-free blood such as serum and plasma, urine, saliva, milk and cerebrospinal fluid sample.

As used herein, the term “blood sample” refers to serum, plasma, cell-free blood, whole blood and its components, blood derived products or preparations. Plasma and serum are very useful as shown in the examples.

The term “quantifying” or “quantification” as used herein refers to absolute quantification, i.e. determining the amount of the respective miRNA but also encompasses measuring the level of the respective miRNA and comparing said level with reference or control miRNA, or comparative expression to other quantified miRNA. Quantification of the respective miRNA as listed in the tables herein allow expression profiling of samples and thus allow identification of signatures associated with diseased samples, as well as identification of signatures associated with prognosis and response to treatment. The quantity of miRNAs or difference in miRNA levels can be determined by any of the methods described herein.

A “control”, “control sample”, or “reference value” or “reference level” are terms which can be used interchangeably herein, and are to be understood as a sample or standard used for comparison with the experimental sample. The control may include a sample obtained from a healthy or non-diseased subject or a subject, which is not at risk of or suffering from bovine tuberculosis. Reference level specifically refers to the level of miRNA or miRNA expression quantified in a sample from a healthy subject, from a subject, which is not at risk of or suffering from bovine tuberculosis. Specifically, a more than 1.3, 1.4, 1.5, 1.6, 1.7, 1.8, 1.9 or 2.0 fold difference between the reference level of one or more miRNAs as defined herein obtained from a sample of a subject. Additionally, a control may also be a standard reference value or range of values, i.e. such as stable expressed miRNAs in the samples, for example the endogenous control.

“Animal(s)”, as used herein, unless otherwise indicated, refers to an individual animal that is a mammal. Specifically, mammal refers to a vertebrate animal that is human and non-human, which are members of the taxonomic class Mammalia. Non-exclusive examples of non-human mammals include companion animals. Non-exclusive examples of a companion animal include: dog, cat, and horse, cows, ferrets, rabbits, pigs, rats, mice, gerbils, hamsters, goats, and the like. Domestic dogs and cats are particular non-limiting examples of pets. Non-exclusive examples of livestock include cattle, sheep, goats, pigs, horses, donkeys, llamas, alpacas, camels, and related domesticated farm animals. Cattle, sheep, and pigs are particular non-limiting examples of livestock species. The term “animal” or “pet” as used in accordance with the present disclosure can further refer to wild animals, including, but not limited to bison, elk, deer, venison, duck, fowl, fish, and the like. As used herein, the terms ‘wild animal’ or ‘wildlife’ are used interchangeably and refer to free-ranging, non-domesticated species that can serve as hosts for Mycobacterium bovis, including, but not limited to, badgers (Meles meles), deer species such as red deer (Cervus elaphus) and white-tailed deer (Odocoileus virginianus), wild boar (Sus scrofa), elk (Cervus canadensis), bison (Bison bison), African buffalo (Syncerus caffer), and other susceptible wildlife reservoirs. In certain embodiments, the wild animal is a badger or a deer species

As used herein, the terms “dog” or “canine” are used interchangeably and refer to any member of the Canidae family including, but not limited to, Canis lupus, Canisfamiliaris, Canis latrans, Canis dingo, Lycaon pictus, Chrysocyon brachyurus, Atelocynus microis, Cuon alpinus, Speothos venaticus, Nyctereutes procyonoides, Vulpes vulpes, and Alopex lagopus. In certain embodiments, the dog or canine is Canisfamiliaris. As used herein, the terms ‘cow’, ‘cattle’ or ‘bovine’ are used interchangeably and refer to any member of the Bovidae family within the genus Bos, including, but not limited to, Bos taurus, Bos indicus, Bos primigenius, and their hybrids. In certain embodiments, the cow or bovine is Bos taurus.

### II. Compositions

The present invention provides genomic identifiers for monitoring and diagnosing TB. These can be used as target nucleic acid sequences for diagnosis of TB in a subject. The diagnostic targets can be used for identification of TB in a sample.

One aspect of the present invention is directed to compositions and methods relating to an assay for the diagnosis of TB. In one embodiment of the present invention, an assay that is rapid, reliable, and can be used for detecting the presence of a target indicator in body fluids. It is especially advantageous that an assay in accordance with the present invention can be useful in diagnosing both early and latent bovine tuberculosis.

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as Sambrook et al., 2000, Molecular Cloning: A Laboratory Manual, third edition, Cold Spring Harbor Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well-known and commonly employed in the art. Standard techniques are used for cloning, DNA and RNA isolation, amplification, and purification. Generally enzymatic reactions involving DNA ligase, DNA polymerase, restriction endonucleases and the like are performed according to the manufacturer's specifications.

Provided herein are methods for assessing, monitoring, and diagnosing bovine tuberculosis in a subject, comprising the steps of: (a) determining the level of expression of each of a plurality of miRNAs within a sample from a subject; and (b) using one or more Artificial Intelligence (AI) model to predict the disease condition of the subject.

### A. MicroRNAs

MicroRNAs (miRNAs) are small, non-coding RNA molecules involved in the regulation of gene expression. Recent studies have demonstrated that miRNAs are stable in body fluids and their expression profiles can reflect pathological conditions, making them promising biomarkers of diseases, including parasitic infections (Manzano-Román R., Mol. Biochem. Parasitol., 2012).

Provided herein are miRNA detection assays that utilize expression profiling combined with powerful bioinformatic analysis and AI modelling. The development of a miRNA-based diagnostic assay for bovine tuberculosis would offer several advantages over current diagnostic methods. The analysis of the expression level of specific miRNAs (Table 1) would enable early detection of bovine tuberculosis while minimizing the incidence of false positives and negatives. The focus on extracellular miRNAs allows the use of biofluid samples (e.g. blood) in a simple laboratory protocol, in conjunction with bespoke and accurate result modelling. MiRNA-based assays can be more cost-effective and less invasive compared to imaging techniques, providing a reliable and accessible diagnostic tool to diagnose and monitor bovine tuberculosis, enabling the ability to detect early or latent infections and complicating disease control efforts.

Nucleotide sequences of mature miRNAs and their respective precursors are known in the art and available from the database miRBase or from the Sanger database.

Identical polynucleotides as used herein in the context of a polynucleotide to be detected by the method as described herein may have a nucleic acid sequence with an identity of at least 90%, 95%, 97%, 98% or 99% or less than 3 or 2 single nucleotide modifications compared to a polynucleotide comprising or consisting of a reference nucleotide sequence of any one of SEQ ID NOs: 1-28.

Furthermore, identical polynucleotides as used herein in the context of a polynucleotide to be detected by the method as described herein may have a nucleic acid sequence with an identity of at least 90%, 95%, 97%, 98% or 99% to a polynucleotide comprising or consisting of the nucleotide sequence of any one of SEQ ID NOs: 1-28 including one, two, three or more nucleotides of the corresponding pre-miRNA sequence at the 5′end and/or the 3′end of the respective seed sequence.

All of the specified miRNAs used according to the invention also encompass isoforms and variants thereof. For the purpose of the invention, the terms “isoforms and variants” (which have also be termed “isomirs”) of a reference miRNA include trimming variants (5′ trimming variants in which the 5′ dicing site is upstream or downstream from the reference miRNA sequence; 3′ trimming variants: the 3′ dicing site is upstream or downstream from the reference miRNA sequence), or variants having one or more nucleotide modifications (3′ nucleotide addition to the 3′ end of the reference miRNA; nucleotide substitution by changing nucleotides from the miRNA precursor), or the complementary mature microRNA strand including its isoforms and variants (for example for a given 5′ mature microRNA the complementary 3′ mature microRNA and vice-versa). With regard to nucleotide modification, the nucleotides relevant for RNA/RNA binding, i.e. the 5′-seed region and nucleotides at the cleavage/anchor side are excluded from modification.

In the following, if not otherwise stated, the term “miRNA” encompasses 3p and 5p strands and their isoforms and variants.

The plurality of miRNAs form a panel comprising the following: mir155, mir17, mir146a, mir16, mir1, mir22, mir 31, mir29a, mir218, mir142, mir 150, mir21, mir194, mir423, mir484, let7a, let7b, mir15b, mir99b, mir106b, mir146b, mir148a, mir193a, mir320a, mir320b, mir374a, mir21, and mir194 as set out below in Table 1.

Measurement of biological signals, including but not limited to miRNA levels derived from quantitative PCR, dPCR, and ddPCR commonly exhibit variability that is unrelated to true biological differences. Such variability may arise from many technical factors. Accordingly, it is often desirable to apply one or more normalization procedures to improve comparability across samples.

In some embodiments, the method further comprises the use of at least one normalizer and/or an off-species control miRNA molecule. In some such embodiments, at least one normalizer is used to ‘normalize’ data, i.e. to control for variation between the samples tested in the method of the invention, and the at least one control is used to try to ensure there are no failure or false readings in the results. An off-species control is added in to show that the miRNAs detected are relevant to the species panel. The off-species control is a miRNA from another species, i.e. not a ruminant. Advantageously, the use of an off-species controls provides another layer of control to distinguish between background or non-specific signals and a positive result. The sequences of the normalizers and the off-species controls that were used are provided below in Table 1.

A variety of normalization techniques are known and may be employed depending on a variety of factors, including study design (Mayer, et al., “Normalization strategies for microRNA profiling experiments: a ‘normal’ way to a hidden layer of complexity?” Biotechnol Lett 32:1777-1788 (2010)). In some embodiments, normalization may be performed using pairwise ratios, mean expression value normalization, or independent standardization. In some embodiments, the present invention may be carried out without need for normalization.

In some embodiments, normalization may be performed using pairwise ratios between measured features (e.g., ratios between expression values of two miRNA molecules). Ratio-based approaches can reduce sensitivity to multiplicative scaling effects because common sample-level factor may cancel in the numerator and denominator. In some embodiments, ratios are computed directly (e.g., x/y). In other embodiments, ratios are computed in log space (e.g., log (x/y)). Pairwise ratios may include use of a fixed referenced feature or use of selected feature pairs.

In some embodiments, normalization may be performed by applying a global scaling factor to measurements within a sample, such as scaling based on a summary statistic such as mean, median, trimmed mean, or geometric mean.

In some embodiments, normalization may include ΔCq (also referred to as ΔCt) processing, in which a quantification cycle value for a target is normalized to a reference within the same sample according to Equation 1 below.

$$Δ Cq = Cq \{ target \} - Cq \{ reference \} \qquad ( Equation 1 )$$

In some embodiments, the normalized values may be further processed using ΔΔCq (ΔΔCt) relative quantification, for example by comparing ΔCq values of a sample to those of a calibrator or control condition. This approach is robust to sample-level technical effects.

In some embodiments, normalization may include independent standardization of one of the features across a cohort of samples. In this type of normalization, values across sample are centered and scaled according to a feature-specific mean and standard deviation.

One of ordinary skill in the art will recognize that any of the forgoing normalization approaches, both alone or in combination, may be applied to the measured data described herein. Unless otherwise required by context, the normalization approaches described herein are exemplary and non-limiting, and equivalent or analogous normalization procedures may be substituted without departing from the scope of the disclosure.

It is preferred that the method comprises the step of assessing the relative levels of miRNA expression of each one of miRNA molecules: mir155, mir17, mir146a, mir16, mir1, mir22, mir 31, mir29a, mir218, mir142, mir 150, mir21, mir194, mir423, mir484, let7a, let7b, mir15b, mir99b, mir106b, mir146b, mir148a, mir193a, mir320a, mir320b, mir374a, mir21, and mir194 within a sample from a subject and using the data obtained from measurement of the expression levels to determine the presence or absence of disease in a subject.

The plurality of target miRNAs form a panel comprising the following: mir155, mir17, mir146a, mir16, mir1, mir22, mir 31, mir29a, mir218, mir142, mir 150, mir21, mir194, mir423, mir484, let7a, let7b, mir15b, mir99b, mir106b, mir146b, mir148a, mir193a, mir320a, mir320b, mir374a, mir21, and mir194.

In some embodiments, the method comprises the step of assessing the relative levels of miRNA expression of a subset of each one of miRNA molecules: mir155, mir17, mir146a, mir16, mir1, mir22, mir 31, mir29a, mir218, mir142, mir150, mir21, mir194, mir423, mir484, let7a, let7b, mir15b, mir99b, mir106b, mir146b, mir148a, mir193a, mir320a, mir320b, mir374a, mir21, and mir194 within a sample from a subject and using the data obtained from measurement of the expression levels to determine the presence or absence of disease in a subject. In some embodiments, the subset is any combination of miR423a, miR320b, miR320a, let7a, miR22, miR150, miR-17, miR-16, miR-22, miR-128, miR-148a, miR-320a, miR-320b, miR-21, miR-423, and miR-193a. In some embodiments, the subset is any combination of miR-17, miR-16, miR-22, miR-128, miR-148a, miR-320a, miR-320b, miR-21, miR-423, and miR-193a. In some embodiments, the subset is any combination of miR423a, miR320b, miR320a, let7a, miR22, and miR150. In some embodiments, the subset is mir-21, mir-423, mir128, mir-17, and mir-16. In some embodiments, the subset is mir-21, mir-423, mir128, mir-17, and mir-16. In some embodiments, the subset is mir-21, mir-423, mir128, mir-17, and mir-16, wherein mir-21 and mir-423 are normalizers.

In some embodiments, at least three miRNA molecules of the forgoing list are assessed. In some embodiments, at least three miRNA molecules and two normalizer miRNA molecules of the forgoing list are assessed.

### III. Methods of Detection

### A. Bovine Tuberculosis

Current methods for diagnosing bovine TB often produce false positives and negatives, requiring the need for multiple tests that can delay results, affecting the ability to detect early or latent infections and complicating disease control efforts.

### 1. Identification of Bovine Tuberculosis

Bovine tuberculosis (bovine TB) is caused by Mycobacterium bovis, which is an aerobic bacterium. Mycobacterium tuberculosis, the bacterium which causes tuberculosis in humans, is closely related. Ingestion or inhalation of the bacterium may cause infection in animals. A range of mammals can be infected by M. bovis, including cattle, humans, deer, llamas, pigs, domestic cats, and wild carnivores and omnivores. It rarely affects sheep. M. bovis is most often transmitted to humans by consumption of raw milk from infected cows. Pasteurization kills M. bovis in infected milk.

In the UK, infected or suspected infected cattle are required by the Department for Environment, Food, and Rural Affairs to be culled to stop the spread of disease to humans. Although cattle can be vaccinated against TB with the BCG vaccine (Fromsa, et al. Science 383(6690): 1-8 (2024)), it is not common, especially in the UK because vaccine can cause false positive results for the current TB test. Unfortunately, the disease continues to persist in many countries, despite efforts to control it.

The tuberculin skin test (TST) was one of the first methods used to diagnose bovine TB and is widely used across the world. It measures cell-mediated immune response following an intradermal skin test with the poorly defined and highly variable tuberculin skin test (TST) antigen. More recently, an in vitro interferon-γ release assay (IGRA) has been introduced as an ancillary test in order to improve the overall sensitivity of detection of bTB-infected animals (EFSA Panel on Animal Health and Welfare, Scientific Opinion on the use of a gamma interferon test for the diagnosis of bovine tuberculosis. EFSA Journal 10(12):2975 (2012); Wood, et al., Tuberculosis 81:147-155 (2001)). The poorly standardized stimulating antigens in the TST (“purified protein derivative” or PPD) are extracts obtained from the heat-killed cultures of specified strains of mycobacteria grown on glycerol broth (Good, et al., Frontiers in Veterinary Science 5(59):1-16 (2018); Yang, et al., FEMS immunology and medical microbiology 66:273-280 (2012)). For instance, bovine PPD (PPD-B) is derived from an extract of M. bovis AN5 strain culture, while avian PPD (PPD-A) is a similarly prepared extract from M. avium subsp. avium D4ER (OIE. Manual of diagnostic Test and Vaccines for Terrestrial Animals. World Organization for Animal Health (2009)). In regions with high exposure to environmental mycobacteria, the difference in increase in skin induration reaction between bovine and avian PPD (i.e. PPD B-A) is ascertained using the single intradermal comparative cervical tuberculin test (SICCT) to improve test specificity, but this is also known to reduce assay sensitivity (de la Rua-Domenech et al., Research in Veterinary Science 81:190-210 (2006)).

Furthermore, the presence of cross-reactive antigens between the pathogenic and vaccine strains in the crude whole cell antigen preparation renders the PPD-based TST unable to differentiate infected from bacille Calmette-Guérin (BCG) vaccinated animals, thereby limiting opportunities for the development of BCG vaccination-based control programs (Brosch, et al., PNAS 104:5596-5601 (2007); Calmette et al., Ann. Inst. Pasteur 50:599-603 (1933); Waters, et al., Vaccine 30:2611-2622 (2012); Young, et al., Science 284, 1479 (1999)).

Bovine tuberculosis can infect animals including cattle, buffaloes, bison, sheep, goats, equines, camels, pigs, wild boars, deer, antelopes, dogs, cats, foxes, mink, badgers, ferrets, rats, primates, llamas, kudus, elands, tapirs, elks, elephants, sitatungas, oryxes, addaxes, rhinoceroses, possums, ground squirrels, otters, seals, hares, moles, raccoons, coyotes, lions, tigers, leopards, lynx, and other felines (Bovine tuberculosis, Oie).

### B. Identification of Target Sequences

The present invention relates to a method for detecting the presence or amount of a target polynucleotide (nucleic acid sequence) from the host's response to bovine tuberculosis in a sample. The target polynucleotide is a virulence determinant. In a preferred embodiment, the target polynucleotide is miRNA. The invention is also directed to a method of detecting the presence of a disease or infection state in a mammal, by detecting the presence or amount of a target miRNA, wherein the presence or amount of the target miRNA identifies the disease state. Thus, the invention relates to diagnostic compositions and methods for detecting bovine tuberculosis. The sample containing the target miRNA may be tissue, collection of cells, cell lysate, body fluid, excretum, in vitro culture, purified polynucleotide, isolated polynucleotide, food sample, medical sample, agro-livestock sample, or environmental sample.

In another embodiment, the invention provides a method for capturing, detecting, and quantifying miRNA from its reverse transcribed cDNA. miRNA is extracted from the provided biological sample using commercially available miRNA specific extraction kits and the manufacturer's recommended protocol (e.g. Qiagen miRNeasy Serum/Plasma Kit). From the extracted miRNA, cDNA is reverse transcribed and amplified using commercially available miRNA to cDNA specific extraction kits and the manufacturer's recommended protocol (e.g. TaqMan Advanced miRNA cDNA Synthesis Kit). The resulting reverse transcribed cDNA of the miRNA may be captured and/or detected using the universal sequences added at both the 5′ and 3′ ends and the cDNA product may undergo universal pre-amplification and/or amplification using a single pair of universal forward and reverse primers. The relative expression levels of specific miRNA, which form part of the defined diagnostic panel, are inferred through the relative expression levels of their respective cDNA, i.e. detection by proxy. This can be performed via numerous traditional DNA detection methods, such as qPCR, digital PCR (dPCR), droplet digital PCR (ddPCR) or Next Generation sequencing, or via newer multiplexing techniques such as beads capture technologies such as the Luminex xMAP system.

The invention described here utilizes large-scale identification of disrupted genes and the use of bioinformatics and AI to select mutants that could be characterized in animals.

C. Multiplex miRNA Profiling

The present invention uses multiplex miRNA relative expression profiling with machine learning and predictive classification analysis. Accuracy of miRNA expression profiling is enhanced when marker expression is analyzed relative to all other markers, as this allows detection of both increased and decreased expression, something not possible with simple threshold level analysis. The present invention used an RT-qPCR approach, with an option step of detection form synthesized cDNA to enhance sensitivity of detection. In one embodiment, the assay uses sample RNA extraction (e.g. Qigen miRNeasy kits), cDNA synthesis and amplification (e.g. TaqMan™ Advanced miRNA cDNA Synthesis Kit) followed by RT-qPCR detection with marker specific primers (e.g. TaqMan™ Advanced miRNA assays). Detection is carried out using a QuantStudio 5 Real-Time PCR Systems, or other RT-qPCR compatible device, to detect miRNA molecules that emit fluorescence that is proportional to their abundance in the samples. An alternate embodiment may utilize alternative PCR technologies such as, but not exclusive to, digital PCR (dPCR), droplet digital PCR (ddPCR). An alternate embodiment may utilize Luminex xMAP system beads, which enable the multiplex capture of miRNAs with picomolar sensitivity and high specificity. The Luminex xMAP beads function with bespoke probes which contain three distinct functional regions: a complementary DNA section to the relevant DNA tag on the Luminex xMAP bead; an RNA region complimentary to the target miRNA; and a biotin fluorescent reporter tag. Detection is carried out using a Luminex LX200, or other compatible device, to detect miRNA molecules that emit fluorescence that is proportional to their abundance in the sample. Each miRNA that was used was given a unique bead region (up to 80 different regions were possible). The data that was obtained from the mixture of particles could then be attributed to the miRNAs by identification of the code.

The disease is selected from the group consisting of bovine tuberculosis and related conditions.

The sample or blood sample refers to tissue or organ sample, blood, cell-free blood such as serum and plasma, urine, saliva, milk and cerebrospinal fluid sample.

From the results of the experiments below, a differentiation in expression levels of miRNA was identified when comparing healthy animals with animals that have bovine tuberculosis. In this instance, infection with bovine tuberculosis was confirmed by a positive SICCT and confirmatory lesions at post mortem with Mycobacterium bovis isolated by culture from these lesions.

### D. Predictive Modeling

Provided herein are methods using predictive modelling to investigate the scope to use the miRNA profiles to predict the presence or absence of disease. A group of healthy and unhealthy animals were taken and tested to determine the level of miRNA expression in samples from these animals. The data obtained was then used to train the models.

Fifteen machine learning models were fitted and compared with the aim of obtaining the best predictions of the disease outcome. Formal assessment of performance was conducted by computing a number of performance statistics based on 5-time repeated 10-fold cross-validation. Cross-validation was useful to obtain more realistic model performance measures from the training data.

Data from the RT-qPCR analysis from each of the miRNA molecules from Table 1 was fitted to each of the models.

This disclosed invention utilized microRNA (miRNA) expression profile analysis to distinguish bovine tuberculosis cases from healthy controls in canine serum samples. A proprietary bioinformatic pipeline incorporating machine learning was used to optimize predictive classification models, achieving in one embodiment an overall accuracy of 70%, sensitivity: 0.77, specificity: 0.6, and ROC AUC: 0.88, and in another embodiment an overall accuracy of 84%, sensitivity: 0.85, specificity: 0.83, and ROC AUC: 0.90. Additionally, principal component analysis (PCA) and clustering confirmed that bovine tuberculosis-associated miRNA profiles were distinct from controls but similar between urine and serum, promoting urine as an additional sample medium. The disclosed methods successfully demonstrated that miRNA biomarkers can differentiate bovine tuberculosis from healthy cases and are detectable in urine.

### IV. Methods of Diagnosing and Treating

In some embodiments, the disclosure provides for a method of diagnosing and treating a subject exhibiting symptoms of bovine tuberculosis or suspected of having bovine tuberculosis, comprising: obtaining a sample from the subject, determining a level of expression of at least one target miRNA by performing an amplification reaction on three or more target miRNA selected from the group consisting of miRNAs: mir155, mir17, mir146a, mir16, mir1, mir22, mir 31, mir29a, mir218, mir142, mir150, mir21, mir194, mir423, mir484, let7a, let7b, mir15b, mir99b, mir106b, mir146b, mir148a, mir193a, mir320a, mir320b, mir374a, mir21, and mir194; applying one or more predictive classification models to the expression of the at least one target miRNA;
using the predictive classification models to diagnose bovine tuberculosis in the subject; and treating the bovine tuberculosis detected in the subject with a treatment for bovine tuberculosis.

In some embodiments, a subset of the above-disclosed group of miRNAs is amplified. In some embodiments, the subset is any combination of miR423a, miR320b, miR320a, let7a, miR22, miR150, miR-17, miR-16, miR-22, miR-128, miR-148a, miR-320a, miR-320b, miR-21, miR-423, and miR-193a. In some embodiments, the subset is any combination of miR-17, miR-16, miR-22, miR-128, miR-148a, miR-320a, miR-320b, miR-21, miR-423, and miR-193a. In some embodiments, the subset is any combination of miR423a, miR320b, miR320a, let7a, miR22, and miR150. In some embodiments, the subset is mir-21, mir-423, mir128, mir-17, and mir-16. In some embodiments, the subset is mir-21, mir-423, mir128, mir-17, and mir-16. In some embodiments, the subset is mir-21, mir-423, mir128, mir-17, and mir-16, wherein mir-21 and mir-423 are normalizers.

In some embodiments, the treatment for bovine tuberculosis is an antibiotic. Antibiotics that may be used in treating tuberculosis include, but are not limited to, ethambutol, isoniazid, pyrazinamide, rifabutin, rifampin, rifapentine, amikacin, capreomycin, cycloserine, ethionamide, levofloxacin, moxifloxacin, para-aminosalicylic acid, and streptomycin. Typically, several antibiotics are administered simultaneously to treat active tuberculosis, whereas a single antibiotic is administered to treat latent tuberculosis. Treatment may continue for at least a month or several months, up to one or two years, or longer, depending on whether the tuberculosis infection is active or latent. Longer treatment is generally required for severe tuberculosis infection, particularly if the infection becomes antibiotic resistant. Latent tuberculosis may be effectively treated in less time, typically 4 to 12 months, to prevent tuberculosis infection from becoming active. Subjects, whose infection is antibiotic resistant, may be screened to determine antibiotic sensitivity in order to identify antibiotics that will eradicate the tuberculosis infection. In addition, corticosteroid medicines also may be administered to reduce inflammation caused by active tuberculosis (see U.S. Pat. No. 10,920,275).

In some embodiments, the disclosure also provides for a method of determining a prognosis for a subject having bovine tuberculosis. In some embodiments, the disclosure also provides for a method of distinguishing active tuberculosis from latent tuberculosis. In some embodiments, the disclosure also provides for a method of monitoring the efficacy of a therapy for treating bovine tuberculosis in a subject.

### V. Kits

Also provided herein is a kit for use in performing the method of the first aspect comprising means for determining the level of expression of each one of the following miRNA molecules: mir155, mir17, mir146a, mir16, mir1, mir22, mir 31, mir29a, mir218, mir142, mir 150, mir21, mir194, mir423, mir484, let7a, let7b, mir15b, mir99b, mir106b, mir146b, mir148a, mir193a, mir320a, mir320b, mir374a, mir21, and mir194. In other embodiments, provided herein is a kit for use in performing the method of the first aspect comprising means for determining the level of expression of each one of the following miRNA molecules: miR-17, miR-16, miR-22, miR-128, miR-148a, miR-320a, miR-320b, miR-21, miR-423, miR-193a. In some embodiments, provided herein is a kit for use in performing the method of the first aspect comprising means for determining the level of expression of each one of the following miRNA molecules: miR-128, miR-17 miR-16, miR-21, and miR-423.

There is also provided a method of selecting a panel for use in disease diagnosis comprising the steps of: (a) selecting a group of miRNA molecules the differential expression of which may be associated with a disease condition; (b) applying one or more predictive classification algorithms to be able to predict the disease condition; and (c) using the one or more predictive classification algorithms to reduce the number of miRNAs in the panel to a minimum number to provide a panel of miRNAs that still produces a result.

For any method disclosed herein that includes discrete steps, the steps may be conducted in any feasible order. And, as appropriate, any combination of two or more steps may be conducted simultaneously.

The description exemplifies illustrative embodiments. In several places throughout the application, guidance is provided through lists of examples, which examples can be used in various combinations. In each instance, the recited list serves only as a representative group and should not be interpreted as an exclusive list.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### VI. Systems and Computer-Implemented Methods

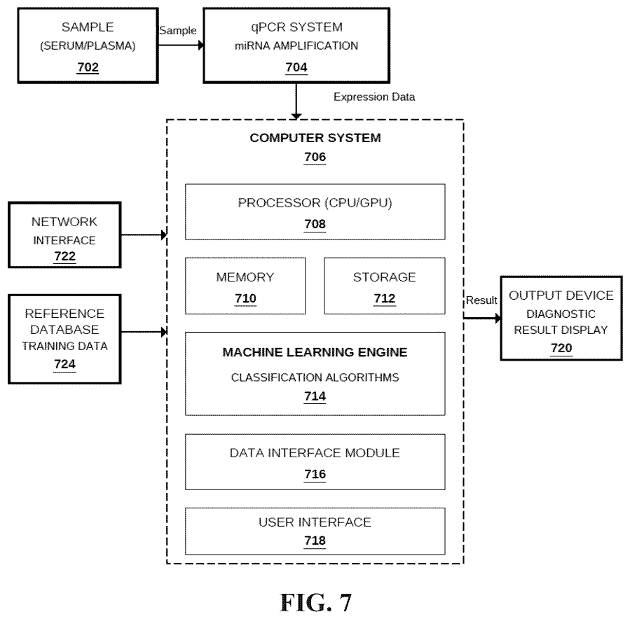

Referring to FIG. 7, which illustrates a diagnostic system for detecting Mycobacterium bovis infection from a bovine sample 702 comprising serum, plasma, or whole blood. The biological sample contains microRNA molecules that serve as biomarkers, with the sample collected using standard veterinary procedures and stored at temperatures between negative twenty degrees Celsius and negative eighty degrees Celsius to preserve miRNA integrity. Control samples are defined by negative Single Intradermal Comparative Cervical Tuberculin (SICCT) skin testing and negative tuberculosis farm history, while reactor samples are characterized by positive skin testing and identification of tuberculosis-related lesions at post-mortem examination, as described herein.

The qPCR system 704 amplifies target miRNA molecules including miR-128, miR-17, miR-16, miR-21, and miR-423 from the bovine sample 702. TaqMan assays optimized for bovine miRNA run each assay in technical duplicate, generating quantification cycle values with a fixed fluorescence threshold. A non-endogenous synthetic spike-in control, cel-miR-39, is added at fixed concentration to monitor extraction efficiency. Assays showing no amplification or Cq values exceeding thirty-five are flagged as undetermined.

A processor 708 within computer system 706 executes computational operations for diagnostic classification. The central processing unit or graphics processing unit performs matrix calculations, statistical analyses, and algorithmic processing required for machine learning operations. Multiple processing cores enable parallel processing of different miRNA targets within samples or concurrent processing of multiple samples to increase diagnostic throughput.

Memory 710 stores software instructions and maintains intermediate computational results during processing. In some embodiments, the volatile storage retains calculated delta quantification cycle values for each target miRNA relative to the mean of miR-21 and miR-423 endogenous controls. In other embodiments, the volatile storage retains other normalized or non-normalize miRNA expression values. Random access memory provides sufficient capacity for multiple concurrent sample analyses without processing delays.

The machine learning engine 714 comprises trained classification models configured to process normalized miRNA expression data. The quadratic discriminant analysis model within machine learning engine 714 achieves overall accuracy of 91%, sensitivity of 0.89, specificity of 0.92, and receiver operating characteristic area under the curve of 0.94 on held-out test data. The partial least squares model provides alternative classification with overall accuracy of 87%, receiver operating characteristic area under the curve of 0.89, sensitivity of 0.87, and specificity of 0.88. In one embodiment, both models within machine learning engine 714 were trained using fifteen classification algorithms including logistic regression, linear discriminant analysis, support vector machines, random forest, k-nearest neighbors, naïve Bayes, neural network variants, XGBoost, and partial least squares discriminant analysis. The classification algorithms within machine learning engine 714 process retained miRNA features that passed data filtering requiring measurable data in at least 57% of samples. Bootstrap resampling of test folds within machine learning engine 714 produces confidence intervals for accuracy and area under the curve measurements, confirming stable performance across the available sample size and cross-validation scheme.

The trained classification model may include a discriminant analysis classifier, wherein the discriminant analysis classifier may include a linear discriminant analysis classifier or a partial least squares discriminant analysis classifier. The trained classification model may further include a generalized linear model classifier, which may include a logistic regression classifier, a Bayesian generalized linear model classifier, or a bootstrapped logistic regression classifier. The trained classification model may include a distance-based classifier, such as a k-nearest neighbors classifier. The trained classification model may also include a decision tree classifier, including a recursive partitioning classifier or a conditional inference tree classifier, as well as an ensemble tree classifier, which may include a bagged decision tree classifier or a random forest classifier. In some embodiments, the trained classification model may include a kernel-based classifier, such as a support vector machine classifier, including linear, polynomial, or radial basis function support vector machines. The trained classification model may further include an artificial neural network classifier, such as a feed-forward neural network, or a gradient-boosted decision tree classifier, including an extreme gradient boosting classifier. One of ordinary skill in the art would recognize that any of the above-referenced classifiers could be used in connection with the invention.

The data interface module 716 receives quantitative expression data from qPCR system 704 and, in some embodiments performs normalization operations. Delta quantification cycle values, or otherwise normalized values are calculated by data interface module 716 using the formula delta Cq equals Cq of target miRNA minus the mean of Cq values for the normalizer miRNA molecules. In some embodiments, said normalizer molecules are miR-21 and miR-423. The normalization process within data interface module 716 controls for inter-sample variation in RNA input and reverse transcription efficiency by using endogenous normalizers. In some embodiments, the endogenous normalizers are miR-21 and miR-423, selected because they exhibited the lowest Z-score variation across the cohort and passed stability criteria. Data interface module 716 performs quality checking of raw Cq values for technical outliers and spike-in performance, excluding samples with aberrant spike-in recovery or technical failure prior to downstream filtering. Variable importance analysis within data interface module 716 identifies some miRNA molecules as the most informative discriminators when normalized to other miRNA molecules. In some embodiments, variable importance analysis within data interface module 716 identifies miR-128, miR-17, and miR-16 as the most informative discriminators when normalized to miR-21 and miR-423 through individual receiver operating characteristic curve analysis and model-specific importance measures.

User interface 718 provides interaction capabilities for system operators to initiate diagnostic procedures and review results. The interface accepts sample identification parameters and presents classification outputs in user-readable format. User interface 718 enables selection between the quadratic discriminant analysis model and partial least squares model for classification, or ensemble combination of both approaches for enhanced diagnostic confidence.

Output device 720 displays diagnostic results indicating Mycobacterium bovis infection status as control or reactor classification. The diagnostic result display presents probability scores from the trained classification models along with confidence intervals derived from bootstrap analysis. Output device 720 provides graphical visualization of results including principal component analysis plots showing multivariate separation between control and reactor samples, hierarchical clustering heatmaps demonstrating distinct expression patterns, and receiver operating characteristic curves with area under the curve measurements. The display capabilities of output device 720 include confusion matrices indicating false-positive and false-negative rates consistent with reported sensitivity and specificity values.

Network interface 722 enables connectivity to external laboratory information systems and veterinary databases for sample tracking and result reporting. The interface supports secure transmission of diagnostic results and integration with herd management systems for actionable decision-making regarding infected animals.

In some embodiments, reference database 724 contains training data comprising a case-control cohort of 170 samples with 85 control animals and 85 tuberculosis reactors. The training data within reference database 724 includes serum aliquots stored at temperatures between −20 and −80° C., with all sample handling following standard biosafety and chain-of-custody procedures. Control samples within reference database 724 are characterized by negative Single Intradermal Comparative Cervical Tuberculin skin testing and negative tuberculosis farm history, while reactor samples are defined by positive skin testing and identification of tuberculosis-related lesions at post-mortem examination. The reference database 724 maintains the final analysis set of 74 samples, in some embodiments comprising 27 controls and 47 reactors, and in some embodiments comprising 70 controls and 97 Reactors, after data filtering and quality control exclusions.

Storage 712 provides persistent retention of trained classification models and reference datasets. The non-volatile storage maintains quadratic discriminant analysis and partial least squares models with parameters determined during ten-fold cross-validation repeated five times. Solid-state or hard disk drives store reference datasets comprising control and reactor expression profiles used during model training. In one embodiment, the diagnostic system may be implemented as a point-of-care (POC) device, where the qPCR amplification system 704 and machine learning classification engine 714 are integrated into a portable farm-side diagnostic system requiring minimal equipment, training, and computational infrastructure, thereby enabling rapid diagnostic results at the location of sample collection without need for transmission to distant laboratory facilities.

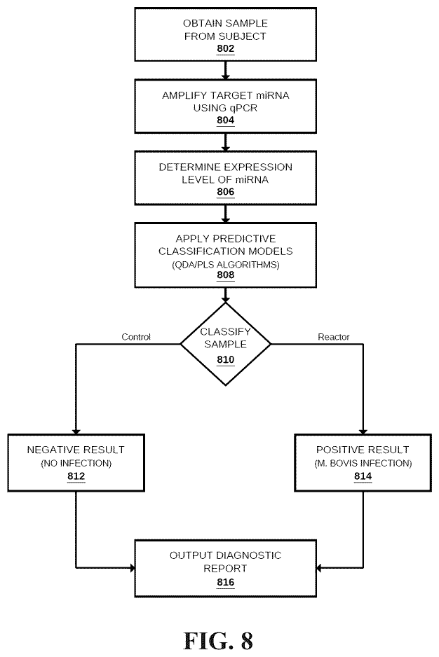

Referring to FIG. 8, a flowchart illustrates a computer-implemented method for detecting Mycobacterium bovis infection in a subject. In step 802, the method includes obtaining a sample from the subject, where the biological sample comprises serum, plasma, or whole blood collected from bovine subjects using standard veterinary procedures. The sample collection process maintains sample integrity through storage at temperatures between −20 degrees Celsius and −80 degrees Celsius until RNA extraction to preserve microRNA molecules that serve as diagnostic biomarkers.

In step 804, the method includes amplifying target miRNA using qPCR, where the amplification targets a panel of microRNA molecules comprising mir155, mir17, mir146a, mir16, mir1, mir22, mir 31, mir29a, mir218, mir142, mir 150, mir21, mir194, mir423, mir484, let7a, let7b, mir15b, mir99b, mir106b, mir146b, mir148a, mir193a, mir320a, mir320b, mir374a, mir21, and mir194, or a subset thereof. In some embodiments, the panel comprises miR-128, miR-17, miR-16, miR-21, and miR-423. The quantitative polymerase chain reaction process employs TaqMan assays optimized for bovine miRNA with each assay run in technical duplicate to ensure measurement reliability. Total RNA enriched for small RNAs is extracted using silica-column kits validated for miRNA recovery, with a non-endogenous synthetic spike-in control cel-miR-39 added at fixed concentration to monitor extraction efficiency and technical variation. Reverse transcription and qPCR generate quantification cycle values recorded using fixed fluorescence threshold, with assays showing no amplification or Cq values exceeding thirty-five flagged as undetermined.

As shown in step 806, the method includes determining expression level of miRNA through quantitative measurement and normalization processes. Raw Cq values undergo quality checking for technical outliers and spike-in performance, with samples showing aberrant spike-in recovery or technical failure excluded prior to downstream analysis. The expression level determination involves calculating delta quantification cycle values using the ΔCq_miRNA=Cq_miRNA−mean (Cq_miR-21, Cq_miR-423), where these endogenous normalizers were selected for exhibiting lowest Z-score variation across cohorts and passing stability criteria.

In step 808, the method includes applying predictive classification models comprising quadratic discriminant analysis and partial least squares algorithms to the normalized expression data. In one example, the trained classification models were developed using fifteen different algorithms including logistic regression, linear discriminant analysis, support vector machines, random forest, k-nearest neighbors, naïve Bayes, neural network variants, XGBoost, and partial least squares discriminant analysis. Model training employed ten-fold cross-validation repeated five times using a reference dataset of, in one embodiment, 74 samples comprising 27 controls and 47 reactors, and in another embodiment, 70 controls and 97 reactors, after data filtering. The quadratic discriminant analysis model achieves overall accuracy of 91%, sensitivity of 0.89, specificity of 0.92, and receiver operating characteristic area under the curve of 0.94, while the partial least squares model provides accuracy of 87% with receiver operating characteristic area under the curve of 0.89.

Continuing with step 810, the method includes classifying the sample based on the output of the predictive classification models. The classification process compares computed probability scores against predetermined threshold values to determine infection status. Bootstrap resampling provides confidence intervals for classification decisions, with calibration analysis confirming that probability outputs align with observed class frequencies across probability bins.

As illustrated in steps 812 and 814, the classification results in either negative results indicating no infection with control status, or positive results indicating Mycobacterium bovis infection with reactor status. Control classifications correspond to subjects with negative Single Intradermal Comparative Cervical Tuberculin (SICCT) skin testing and negative tuberculosis farm history, while reactor classifications indicate subjects with positive skin testing and identification of tuberculosis-related lesions at post-mortem examination.

In step 816, the method includes outputting a diagnostic report containing the classification results and associated confidence measures. The diagnostic report provides actionable information for herd management decisions including treatment protocols or removal procedures for infected animals. The output format supports integration with veterinary information systems and regulatory reporting requirements for tuberculosis surveillance programs.

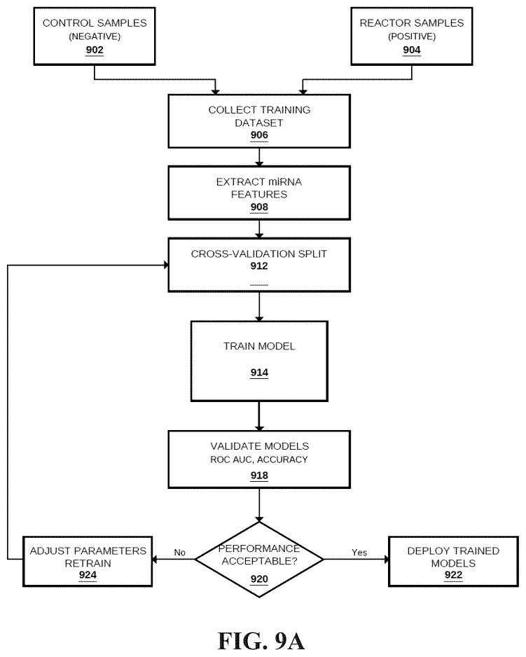

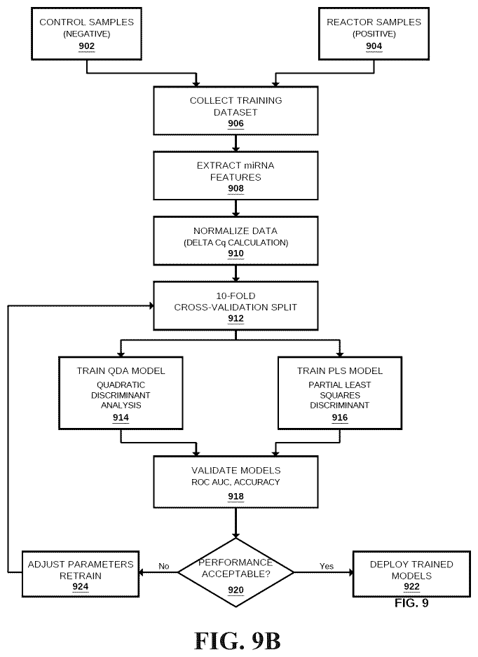

Referring to FIGS. 9A-B, a flowchart illustrates a machine learning model training process for developing classification algorithms to detect Mycobacterium bovis infection. The training process begins with control samples 902 comprising biological samples from subjects without Mycobacterium bovis infection. Control samples 902 represent the negative class data characterized by absence of disease markers, providing baseline miRNA expression patterns for comparison against infected subjects.

The process includes reactor samples 904 comprising biological samples from subjects with confirmed Mycobacterium bovis infection. Reactor samples 904 provide the positive class data representing subjects with disease markers, enabling identification of altered miRNA expression patterns associated with infection status.

As shown in step 906, the method includes collecting a training dataset by assembling control samples 902 and reactor samples 904 into a balanced case-control dataset suitable for supervised machine learning. The training dataset provides the foundational data structure required for developing binary classification models that can distinguish between infected and non-infected subjects based on miRNA biomarker profiles.

Continuing with step 908, the method includes extracting miRNA features from the biological samples in the training dataset. The feature extraction process generates quantitative expression data for a panel of miRNA molecules through amplification and measurement techniques, producing numerical values representing relative abundance of each target miRNA within the samples.

At step 910, the method includes normalizing data by calculating delta quantification cycle values for each target miRNA relative to endogenous control miRNAs. The normalization process controls for inter-sample variation and technical factors that could influence measurement accuracy, producing standardized expression values suitable for machine learning analysis. In some embodiments, the method includes normalizing data by another method as described herein. In some embodiments, the method does not include a normalization step.

As illustrated in step 912, the method includes performing k-fold cross-validation split to partition the training dataset for model development and validation. In certain embodiments, the method includes performing ten-fold cross-validation split. One skilled in the art would recognize that a variety of cross-validation configurations and validation strategies may be used. In some embodiments, the cross-validation is determined based on the size of the data set. The cross-validation methodology creates multiple training and testing partitions to enable robust performance assessment and prevent overfitting during model development.

The process continues at step 914 with training a quadratic discriminant analysis model using the partitioned training data from the cross-validation split. In some embodiments, the quadratic discriminant analysis classifier learns to distinguish between control and reactor samples by identifying optimal decision boundaries in the normalized miRNA expression space, with model parameters optimized through iterative training processes that minimize classification error on the training partitions.

In some embodiments, simultaneously, step 916 includes training a partial least squares model as an alternative classification approach using the same training data partitions. The partial least squares discriminant analysis develops linear combinations of miRNA features that maximize separation between control and reactor classes while reducing dimensionality of the input data, providing a complementary classification methodology to the quadratic discriminant analysis approach.

Following model training, step 918 includes validating models using receiver operating characteristic area under the curve and accuracy metrics computed on held-out test folds from the cross-validation process. The validation process aggregates performance statistics across multiple cross-validation repeats to estimate out-of-sample classification performance, including sensitivity, specificity, overall accuracy, false discovery rate, and area under the receiver operating characteristic curve measurements that quantify the models' discriminative ability. Bootstrap resampling of the test folds generates empirical distributions of performance metrics by resampling test fold predictions with replacement across multiple iterations, from which percentile-based confidence intervals (e.g., 2.5th to 97.5th percentiles) are calculated to establish the 95% confidence bounds for the area under the receiver operating characteristic curve and other key performance measures.

At decision step 920, the training process evaluates whether performance is acceptable by comparing computed validation metrics against predetermined threshold criteria for diagnostic accuracy. The performance assessment determines whether the trained models achieve sufficient discriminative power for clinical deployment based on statistical significance of classification results and confidence intervals derived from bootstrap resampling of test folds.

When performance is acceptable, step 922 includes deploying the trained models for operational use in diagnostic applications. The deployment process transfers the optimized model parameters and associated preprocessing steps to the production diagnostic system, enabling classification of new samples using the validated algorithms.

If performance proves inadequate, step 924 includes adjusting parameters and retraining the models with modified hyperparameters, feature selection, or data preprocessing approaches. The parameter adjustment process returns to earlier training steps to optimize model performance through iterative refinement until acceptable classification accuracy is achieved.

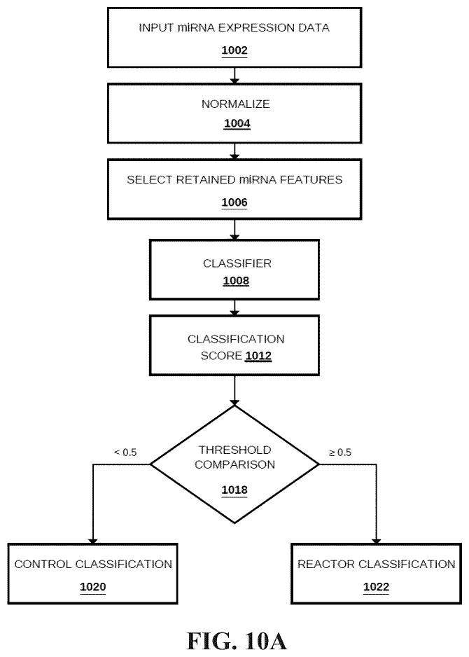

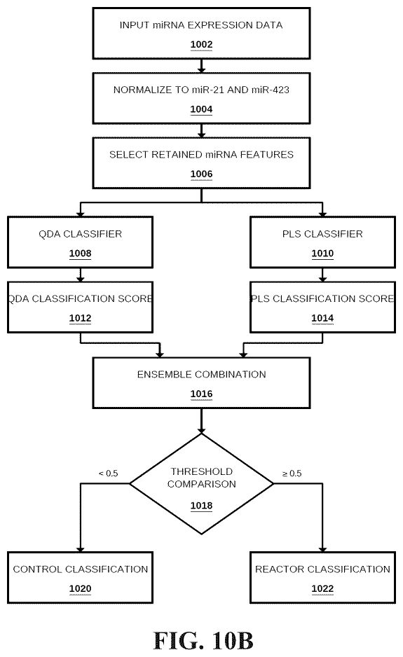

Referring to FIG. 10A-B, a flowchart illustrates the operational classification process for analyzing new samples using trained machine learning models. This illustrates one embodiment of the operational classification process, with exemplary miRNAs represented. The process begins at step 1002 with inputting miRNA expression data comprising quantitative measurements of target miRNA molecules from a biological sample. The input data represents raw quantification cycle values generated through amplification of the miRNA panel from the test sample.

As shown in step 1004, the method, in some embodiments, includes normalizing to miR-21 and miR-423 by calculating delta quantification cycle values for each target miRNA relative to the mean quantification cycle values of these endogenous control miRNAs. As suggested herein, other normalization techniques may be used as descried herein. In some embodiments, no normalization is performed. The normalization process controls for inter-sample variation in RNA input and reverse transcription efficiency, producing standardized expression values suitable for classification analysis.

Continuing with step 1006, the process includes selecting retained miRNA features that correspond to the miRNA molecules used during model training. The feature selection ensures that only validated biomarkers with demonstrated discriminative power are included in the classification analysis, maintaining consistency with the training dataset composition.

At decision point 1008, the normalized, or, in some embodiments, not normalized, expression data is processed by a quadratic discriminant analysis classifier that applies learned decision boundaries to generate a classification probability score. The quadratic discriminant analysis classifier evaluates the normalized miRNA expression pattern against the trained model parameters to compute the likelihood of Mycobacterium bovis infection.

Simultaneously at step 1010, the same normalized expression data is processed by a partial least squares classifier that generates an independent classification probability score using linear discriminant analysis techniques. The partial least squares classifier provides an alternative classification approach that can validate or complement the quadratic discriminant analysis results.

The outputs from both classifiers are processed at step 1012 to generate a quadratic discriminant analysis classification score and at step 1014 to generate a partial least squares classification score. These individual classifier outputs provide quantitative measures of infection probability based on different mathematical approaches to pattern recognition in the miRNA expression data.

In some embodiments, following individual scoring, step 1016 implements ensemble combination that integrates the individual classification scores from both models to generate a composite classification result. The ensemble approach leverages the strengths of both classification methodologies to improve overall diagnostic accuracy and reduce the impact of individual model limitations.

The combined classification score undergoes threshold comparison at step 1018, where scores less than 0.5 direct the process toward control classification 1020, while scores greater than or equal to 0.5 direct toward reactor classification 1022. The threshold comparison provides the final binary classification decision that determines the diagnostic outcome for the test sample.

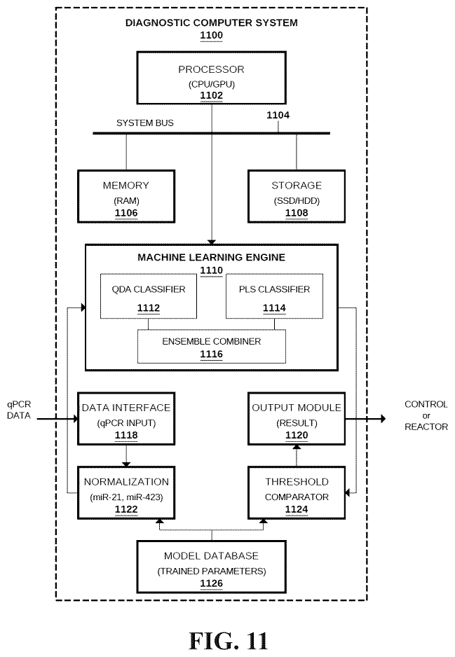

Referring to FIG. 11, a diagnostic computer system 1100 comprises specialized hardware and software components configured to implement machine learning-based classification for detecting Mycobacterium bovis infection. This illustrates one embodiment of the operational classification process, with exemplary miRNAs represented. The diagnostic computer system 1100 processes quantitative polymerase chain reaction data from miRNA amplification and generates binary classification outputs indicating control or reactor status for biological samples.

The processor 1102 comprises a central processing unit or graphics processing unit configured to execute computational operations required for machine learning classification algorithms. The processor 1102 performs matrix calculations, statistical analyses, and algorithmic processing necessary for quadratic discriminant analysis and partial least squares classification models. Multiple processing cores within processor 1102 enable parallel processing of different miRNA targets within samples or concurrent processing of multiple samples to increase diagnostic throughput while maintaining real-time classification capabilities suitable for field deployment.

As shown in the architecture, system bus 1104 provides high-speed data communication pathways connecting processor 1102 with other system components. The system bus 1104 facilitates rapid data transfer between processing units, memory systems, and input-output modules to minimize processing latency during classification operations. Data bandwidth capabilities of system bus 1104 support simultaneous access to multiple system resources during intensive computational operations.

Memory 1106 comprising random access memory provides volatile storage for active data and software instructions during classification processing. The memory 1106 maintains intermediate computational results including calculated delta quantification cycle values for target miRNAs relative to endogenous control miRNAs, which in some embodiment are miR-21 and miR-423. Sufficient memory capacity within memory 1106 enables concurrent processing of multiple samples without memory constraints that would impact classification speed or accuracy.

Storage 1108 provides persistent data storage using solid-state drives or hard disk drives configured to retain trained classification models and reference datasets across system power cycles. The storage 1108 maintains quadratic discriminant analysis and partial least squares models with parameters determined during training using ten-fold cross-validation repeated five times on reference datasets. Long-term data retention capabilities of storage 1108 support archival storage of processed diagnostic results for longitudinal analysis and quality assurance tracking.

The machine learning engine 1110 comprises trained classification algorithms configured to process normalized miRNA expression data from biological samples. The machine learning engine 1110 implements both quadratic discriminant analysis and partial least squares classification approaches developed through training on fifteen different algorithmic approaches including logistic regression, linear discriminant analysis, support vector machines, random forest, k-nearest neighbors, naïve Bayes, neural network variants, XGBoost, and partial least squares discriminant analysis.

Within machine learning engine 1110, the quadratic discriminant analysis classifier 1112 applies learned decision boundaries to normalized expression data for a panel comprising mir155, mir17, mir146a, mir16, mir1, mir22, mir 31, mir29a, mir218, mir142, mir 150, mir21, mir194, mir423, mir484, let7a, let7b, mir15b, mir99b, mir106b, mir146b, mir148a, mir193a, mir320a, mir320b, mir374a, mir21, and mir194, or a subset thereof. In some embodiments, the panel comprises miR-128, miR-17, miR-16, miR-21, and miR-423. The quadratic discriminant analysis classifier 1112 achieves overall accuracy of 91%, sensitivity of 0.89, specificity of 0.92, for the smaller panel, and receiver operating characteristic area under the curve of 0.94 based on validation using held-out test data from cross-validation analysis.

Simultaneously, the partial least squares classifier 1114 provides alternative classification using linear discriminant analysis techniques applied to the same miRNA expression panel. The partial least squares classifier 1114 demonstrates overall accuracy of 87%, receiver operating characteristic area under the curve of 0.89, sensitivity of 0.87, and specificity of 0.88, providing complementary classification results that can validate quadratic discriminant analysis outputs.

The ensemble combiner 1116 integrates classification scores from both the quadratic discriminant analysis classifier 1112 and partial least squares classifier 1114 to generate composite classification results. The ensemble combiner 1116 leverages the discriminative strengths of both classification methodologies to improve overall diagnostic accuracy and reduce individual model limitations through mathematical combination of probability scores.

Data interface 1118 receives qPCR input data comprising raw quantification cycle values from amplification of the target miRNA panel. The data interface 1118 implements quality control procedures including assessment of technical outliers and validation of spike-in control performance to ensure data integrity prior to normalization and classification processing.

Normalization module 1122 calculates delta quantification cycle values using the formula delta Cq equals Cq of target miRNA minus the mean of Cq values for normalization miRNA molecules, which, in some embodiments, are miR-21 and miR-423. The normalization module 1122 controls for inter-sample variation in RNA input and reverse transcription efficiency by using endogenous normalizers selected for exhibiting lowest Z-score variation across validation cohorts and passing stability criteria, which in some embodiments are miR-21 and miR-423.

Output module 1120 generates diagnostic results indicating classification outcomes as control or reactor status based on processed miRNA expression patterns. The output module 1120 provides probability scores, confidence intervals, and associated performance metrics derived from the trained classification models to support clinical decision-making processes.

Threshold comparator 1124 evaluates combined classification scores from ensemble combiner 1116 against predetermined decision thresholds to generate binary classification outputs. The threshold comparator 1124 applies a threshold value of 0.5, directing scores below this threshold toward control classification and scores at or above this threshold toward reactor classification, providing definitive diagnostic determinations for biological samples.

Model database 1126 stores trained parameters for both classification models including decision boundaries, feature weights, and preprocessing parameters determined during the training process. The model database 1126 maintains reference data from the final analysis set comprising seventy-four samples with twenty-seven controls and forty-seven reactors used for model development and validation, enabling consistent application of trained algorithms to new diagnostic samples. The disclosed method has been validated using retrospective cross-validation analysis on historical samples collected from known infected and control animals. Further prospective validation on independently collected samples from animals with unknown infection status would confirm the generalizability and real-world applicability of the classification models under field conditions in diverse herds and geographic locations.

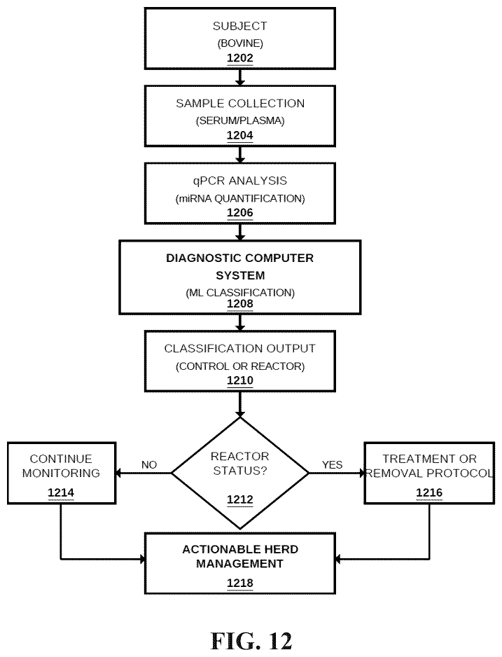

Referring to FIG. 12, a flowchart illustrates the complete diagnostic workflow from subject evaluation through actionable herd management decisions for detecting Mycobacterium bovis infection. The process begins with subject 1202 comprising a bovine animal selected from species susceptible to Mycobacterium bovis infection including cattle, buffaloes, humans, bison, sheep, goats, equines, camels, pigs, wild boars, deer, antelopes, dogs, cats, foxes, mink, badgers, ferrets, rats, primates, llamas, kudus, elands, tapirs, elks, elephants, sitatungas, oryxes, addaxes, rhinoceroses, possums, ground squirrels, otters, seals, hares, moles, raccoons, coyotes, lions, tigers, leopards, lynx, and other felines that may harbor Mycobacterium bovis infections. One skilled in the art will understand that animals having similar genomic characteristics, including humans, may likewise serve as subjects of the present invention.

The workflow continues with sample collection 1204 involving collection of serum or plasma from the bovine subject using standard veterinary procedures. Sample collection 1204 generates biological specimens containing microRNA molecules that serve as biomarkers for tuberculosis infection, with collected samples stored at temperatures between −20° C. and −80° C. to preserve miRNA integrity until processing. The sample collection process follows standard biosafety and chain-of-custody procedures to ensure sample quality and traceability throughout the diagnostic workflow.

Following sample acquisition, qPCR analysis 1206 performs miRNA quantification using amplification of a target panel comprising mir155, mir17, mir146a, mir16, mir1, mir22, mir 31, mir29a, mir218, mir142, mir 150, mir21, mir194, mir423, mir484, let7a, let7b, mir15b, mir99b, mir106b, mir146b, mir148a, mir193a, mir320a, mir320b, mir374a, mir21, and mir194, or a subset thereof from the biological sample. In some embodiments, the panel comprises miR-128, miR-17, miR-16, miR-21, and miR-423. The qPCR analysis 1206 employs TaqMan assays optimized for bovine miRNA with each assay run in technical duplicate to ensure measurement reliability and accuracy. Total RNA enriched for small RNAs undergoes extraction using silica-column kits validated for miRNA recovery, with non-endogenous synthetic spike-in control cel-miR-39 added at fixed concentration to monitor extraction efficiency and technical variation throughout the quantification process.

The quantified expression data is processed by diagnostic computer system 1208 implementing machine learning classification algorithms to determine infection status. The diagnostic computer system 1208 comprises trained quadratic discriminant analysis and partial least squares models that process normalized miRNA expression data to generate probability scores for classification decisions. The quadratic discriminant analysis model within diagnostic computer system 1208 achieves overall accuracy of 91%, sensitivity of 0.89, specificity of 0.92, and receiver operating characteristic area under the curve of 0.94, while the partial least squares model provides complementary classification with accuracy of 87% and receiver operating characteristic area under the curve of 0.89. In an alternative embodiment, the diagnostic computer system 1208 operates in batch processing mode, where the system receives quantitative expression data for a plurality of samples and generates classification outputs for all samples in a single computational cycle, thereby improving diagnostic throughput for herd-level screening and enabling efficient processing of multiple animals from the same facility.

Processing by the diagnostic computer system 1208 generates classification output 1210 indicating either control or reactor status based on the miRNA expression pattern analysis. The classification output 1210 provides quantitative probability scores along with binary diagnostic determinations derived from threshold comparison of ensemble classification results. Control classifications within classification output 1210 correspond to subjects with miRNA expression patterns consistent with absence of Mycobacterium bovis infection, while reactor classifications indicate expression patterns associated with active or latent tuberculosis infection.

At decision point 1212, the diagnostic workflow evaluates reactor status based on the classification output 1210 to determine appropriate management actions. The reactor status evaluation compares the classification results against established diagnostic criteria to direct subsequent animal health management decisions and regulatory compliance requirements.

When reactor status evaluation 1212 indicates negative results, the workflow directs toward continue monitoring 1214 involving ongoing surveillance and periodic retesting protocols. Continue monitoring 1214 maintains the animal within the production system while implementing appropriate biosecurity measures and scheduling follow-up diagnostic assessments according to regulatory guidelines and veterinary recommendations for tuberculosis surveillance programs.

When reactor status evaluation 1212 indicates positive results, the workflow proceeds to treatment or removal protocol 1216 involving therapeutic intervention or culling procedures as dictated by regulatory requirements and animal health management protocols. Treatment options within treatment or removal protocol 1216 include antibiotic therapy using agents such as rifampicin, isoniazid, pyrazinamide, and ethambutol, with treatment regimens continuing for extended periods ranging from several months to over one year depending on infection severity and antibiotic resistance patterns. Alternative management approaches within treatment or removal protocol 1216 include removal of infected animals from production systems through culling procedures mandated by regulatory authorities to prevent disease transmission within herds and to human populations.

The diagnostic workflow culminates with actionable herd management 1218 comprising comprehensive management decisions based on diagnostic results and regulatory requirements. Actionable herd management 1218 includes implementation of movement restrictions, quarantine procedures, contact tracing, and enhanced surveillance protocols for animals within affected herds. The management decisions within actionable herd management 1218 support timely herd-level control measures that minimize unnecessary culling while preventing undetected within-herd transmission and reducing economic and operational burdens on producers and veterinary services. Integration of diagnostic results into herd management information systems enables data-driven decision-making that optimizes animal health outcomes while maintaining regulatory compliance with tuberculosis eradication programs.

### EXAMPLES

The present invention is illustrated by the following examples. It is to be understood that the particular examples, materials, amounts, and procedures are to be interpreted broadly in accordance with the scope and spirit of the invention as set forth herein.

### Example 1: qPCR-based miRNA Profiling with Machine Learning

Optimization

### Materials and Methods

A targeted literature review was performed by MI: RNA Diagnostics to identify circulating bovine microRNAs reported in association with Mycobacterium bovis infection and early-stage tuberculosis. From this review, 32 candidate miRNAs (shown in Table1) were selected based on reported differential expression, reproducibility across studies, and potential biological relevance to host immune response. These 32 miRNAs formed the initial assay panel for experimental evaluation.

TABLE 1

| SEQ ID | | |
|---|---|---|
| NO. | Name | Sequence |
| 1 | mir155 | UUAAUGCUAAUCGUGAUAGGGGU |
| 2 | mir17 | CAAAGUGCUUACAGUGCAGGUAGU |
| 3 | mir146a | UGAGAACUGAAUUCCAUAGGUUGU |
| 4 | mir16 | UAGCAGCACGUAAAUAUUGGUG |
| 5 | mir1 | UGGAAUGUAAAGAAGUAUGUAU |
| 6 | mir22 | AAGCUGCCAGUUGAAGAACUG |
| 7 | mir31 | AGGCAAGAUGCUGGCAUAGCU |
| 8 | mir29a | CUAGCACCAUCUGAAAUCGGUUA |
| 9 | mir128 | UCACAGUGAACCGGUCUCUUU |
| 10 | mir142 | CAUAAAGUAGAAAGCACUAC |
| 11 | mir150 | UCUCCCAACCCUUGUACCAGUGU |
| 12 | mir21 | UAGCUUAUCAGACUGAUGUUGACU |
| 13 | mir194 | UGUAACAGCAACUCCAUGUGGA |
| 14 | mir423 | UGAGGGGCAGAGAGCGAGACUUU |
| 15 | mir484 | UCAGGCUCAGUCCCCUCCCGAU |
| 16 | let7a | UGAGGUAGUAGGUUGUAUAGUU |
| 17 | let7b | UGAGGUAGUAGGUUGUGUGGUU |
| 18 | mir15b | UAGCAGCACAUCAUGGUUUACA |
| 19 | mir99b | CACCCGUAGAACCGACCUUGCG |
| 20 | mir106b | UAAAGUGCUGACAGUGCAGAU |
| 21 | mir146b | UGAGAACUGAAUUCCAUAGGCUGU |
| 22 | mir148a | UCAGUGCACUACAGAACUUUGU |
| 23 | mir193a | UGGGUCUUUGCGGGCGAGAUGA |
| 24 | mir320a | AAAAGCUGGGUUGAGAGGGCGA |
| 25 | mir320b | AGCUGGGUUGAGAGGGUGGU |
| 26 | mir374a | UUAUAAUACAACCUGAUAAGUG |
| 27 | mir21 | UAGCUUAUCAGACUGAUGUUGACU |
| 28 | mir194 | UGUAACAGCAACUCCAUGUGGA |

### Cohort and Sample Collection

A case-control cohort comprising 170 samples was assembled for assay evaluation: 85 control animals and 85 TB reactors: controls as defined by negative SICCT skin testing and a negative TB farm history; positive cases defined by positive SICCT and identification of TB related lesions at post-mortem. Serum aliquots were stored at between −20 and −80° C. until RNA extraction. All sample handling followed standard biosafety and chain-of-custody procedures. Infection with bovine tuberculosis was confirmed by a positive SICCT and confirmatory lesions at post mortem with Mycobacterium bovis isolated by culture from these lesions.

### RNA Extraction and Spike-In Control

Total RNA enriched for small RNAs was extracted from a fixed volume of serum using a silica-column kit validated for miRNA recovery. A non-endogenous synthetic spike-in control (cel-miR-39) was added to each sample prior to extraction at a fixed concentration to monitor extraction efficiency and technical variation. RNA yields and purity were assessed by fluorometric quantification and inspection of extraction controls.

qPCR Assay and Data Acquisition

Reverse transcription and qPCR were performed using TaqMan assays optimized for the panel of bovine miRNA. Each miRNA assay was run in technical duplicate. Cq values were recorded using a fixed fluorescence threshold and exported for downstream analysis. Assays with no amplification or Cq>35 were flagged as undetermined and treated as missing data for that miRNA in that sample.

### Normalization Strategy and Quality Control

Raw Cq values were quality-checked for technical outliers and spike-in performance. To control for inter-sample variation in RNA input and reverse transcription efficiency, delta Cq (ΔCq) values were calculated for each miRNA as:

$$Δ {Cq}_{\{ miRNA \}} = {Cq}_{\{ miRNA \}} - \backslash mathrm \{ mean \} ( {Cq}_{\{ miR 21 \}} , {Cq}_{\{ miR 423 \}} ) \qquad ( Equation 2 )$$

miR-21 (SEQ ID NO: 12) and miR-423 (SEQ ID NO: 14) were selected as endogenous normalizers because they exhibited the lowest Z-score variation across the cohort after initial normalization and passed stability criteria. Samples with aberrant spike-in recovery or technical failure were excluded prior to downstream filtering.

### Data Filtering and Final Analysis Set

miRNA features were retained only if they had measurable data in at least 75% of samples (i.e., features missing in >25% of samples were removed). The 75% sample retention threshold was established to maintain sufficient data density and statistical power for downstream model training while excluding features with excessive missing data that could introduce bias or compromise predictive accuracy. This filtering reduced the panel from 32 candidates to 10 informative miRNAs: miR-17, miR-16, miR-22, miR-128, miR-148a, miR-320a, miR-320b, miR-21, miR-423, miR-193a. After feature filtering, samples missing any of the retained miRNA measurements were excluded, yielding a final analysis set of 74 samples (27 controls, 47 reactors).

### Machine Learning Model Training and Evaluation

A comprehensive screening of fifteen distinct classification algorithms was conducted to identify the optimal discriminative model for control versus reactor classification using the retained miRNA features. This multi-algorithm evaluation approach enabled comparative assessment of algorithm performance and robustness across different statistical and machine learning paradigms. The model set included 13 previously established algorithms (for example logistic regression, linear discriminant analysis, support vector machines, random forest, k-nearest neighbors, naïve Bayes, neural network variants), ensemble methods including XGBoost (a gradient boosting ensemble method) and PLS-DA. All models were trained using 10-fold cross-validation repeated five times with default tuning parameters to estimate out-of-sample performance and reduce variance from fold assignment. Model training and evaluation were implemented in R and/or Python using established libraries; software versions and package names were recorded in the analysis log.

Model performance was assessed using the following metrics computed on held-out folds and aggregated across repeats: ROC AUC, overall accuracy, sensitivity (true positive rate), and specificity (true negative rate). Confidence intervals for ROC AUC were estimated using bootstrap resampling of test folds, a nonparametric resampling approach in which the observed test fold predictions are resampled with replacement to generate empirical distributions of ROC AUC values, from which percentile-based confidence intervals are calculated. The best performing model was identified by a combination of ROC AUC and balanced sensitivity/specificity.

### Variable Importance and Marker Selection

Variable importance and biomarker selection were performed through integrated multivariate and univariate analyses. Individual ROC curve analysis was conducted for each miRNA as a univariate discriminator (area under the single-marker ROC curve), quantifying the standalone predictive capacity of each miRNA. Model-specific importance measures (e.g., feature importance from tree-based models, coefficient magnitude for linear models, and loadings for PLS-DA) were derived from each trained algorithm. Markers demonstrating convergent importance ranking across multiple independent importance estimation methods were identified as the most biologically and statistically informative biomarkers and were selected for minimal panel construction. A minimal panel was evaluated by retraining a PLS model on the selected markers and comparing performance metrics to the full model.

### Results

### Multivariate Separation of Groups

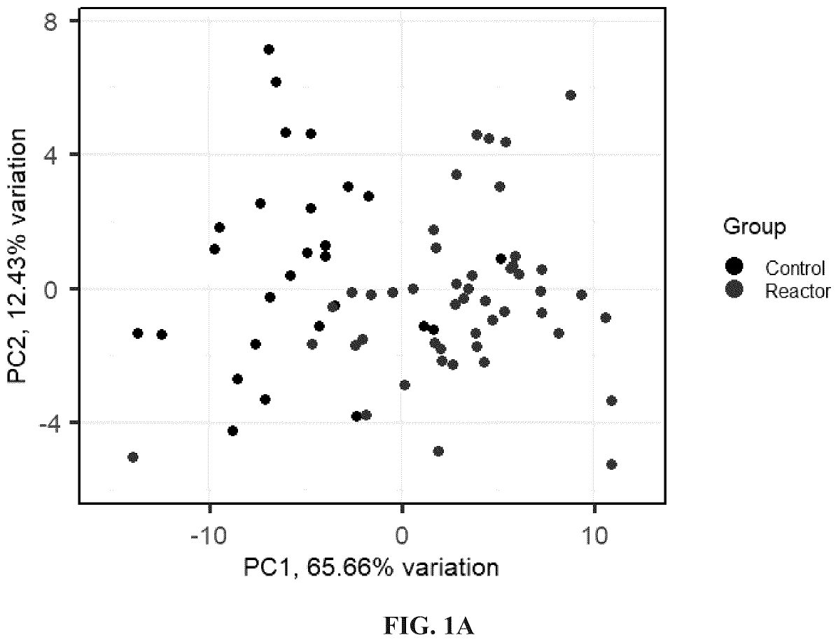

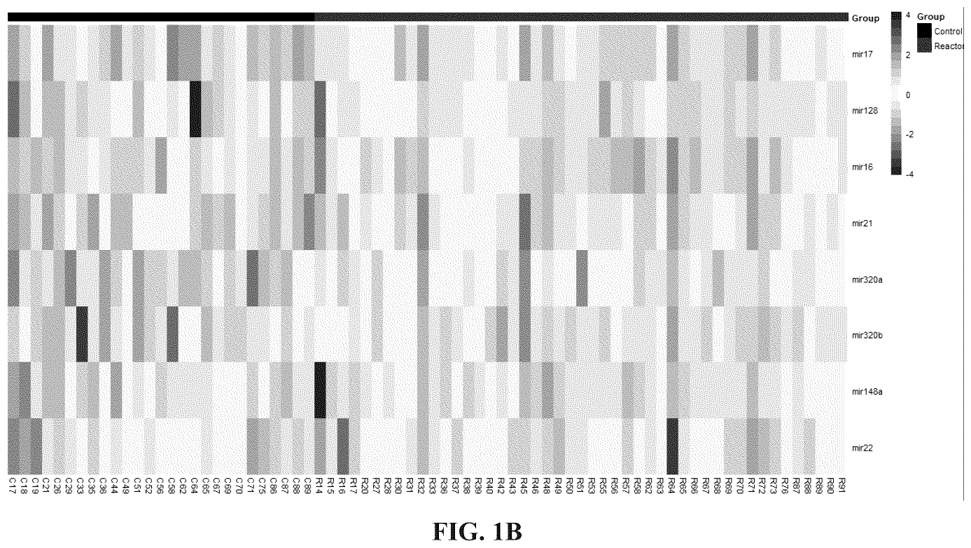

Principal component analysis (PCA) of the normalized qPCR miRNA dataset revealed clear multivariate structure separating control and reactor samples. Control and reactor samples segregated primarily along the first principal component (PC1), which accounted for the largest proportion of variance in the dataset (FIG. 1A). The direction and magnitude of sample loadings on PC1 were consistent with higher or lower expression of a subset of miRNAs in reactors versus controls, indicating a coordinated host miRNA response associated with reactor status.

### Expression Heatmap and Clustering

A hierarchical clustering heatmap of the retained miRNA features corroborated the PCA results. The heatmap shows distinct expression patterns between groups, with clusters of miRNAs up- or down-regulated in reactors relative to controls (FIG. 1B). Sample clustering largely grouped reactors together and controls together, with a minority of samples occupying intermediate positions consistent with biological or technical heterogeneity.

### Classifier Performance-QDA (Lead Model)

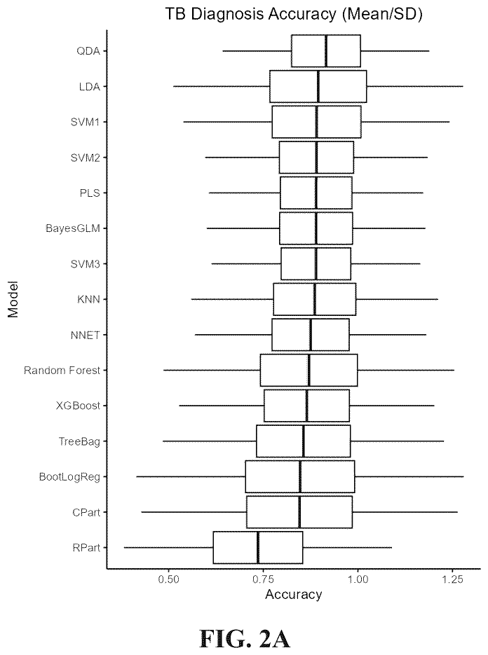

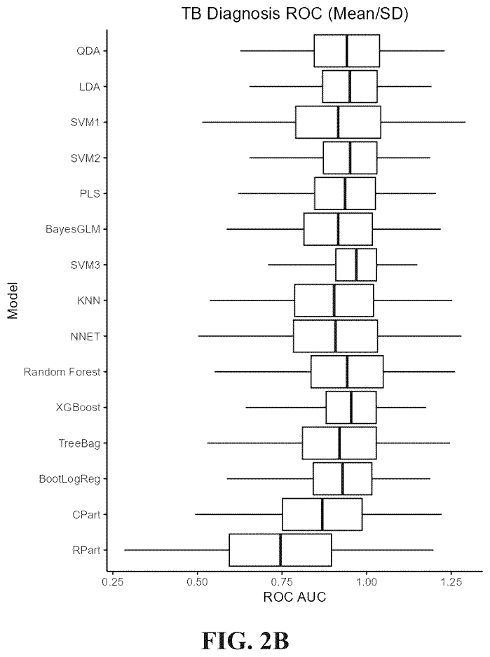

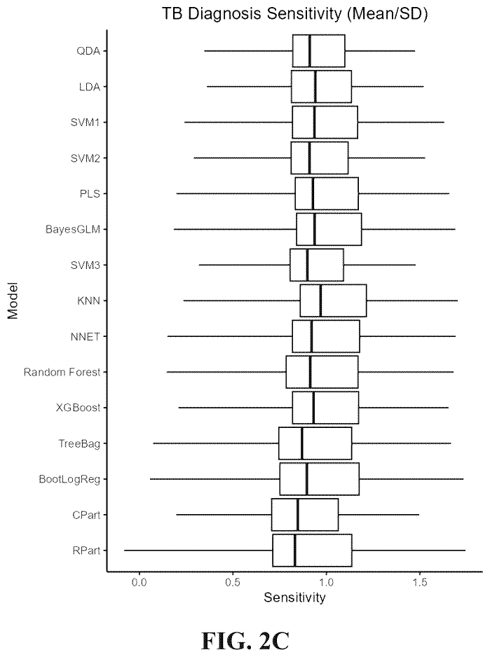

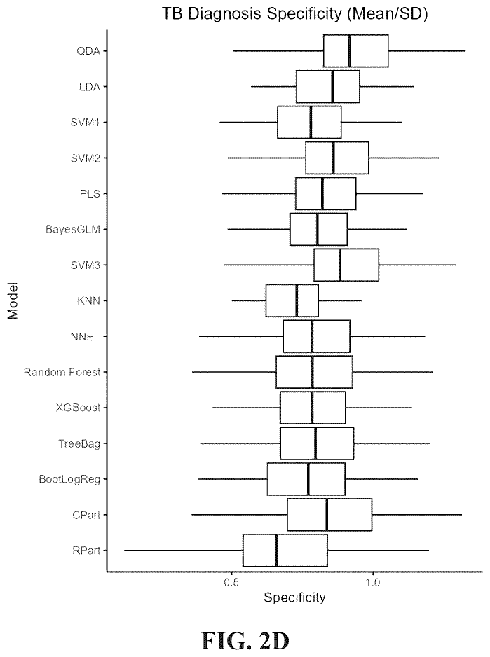

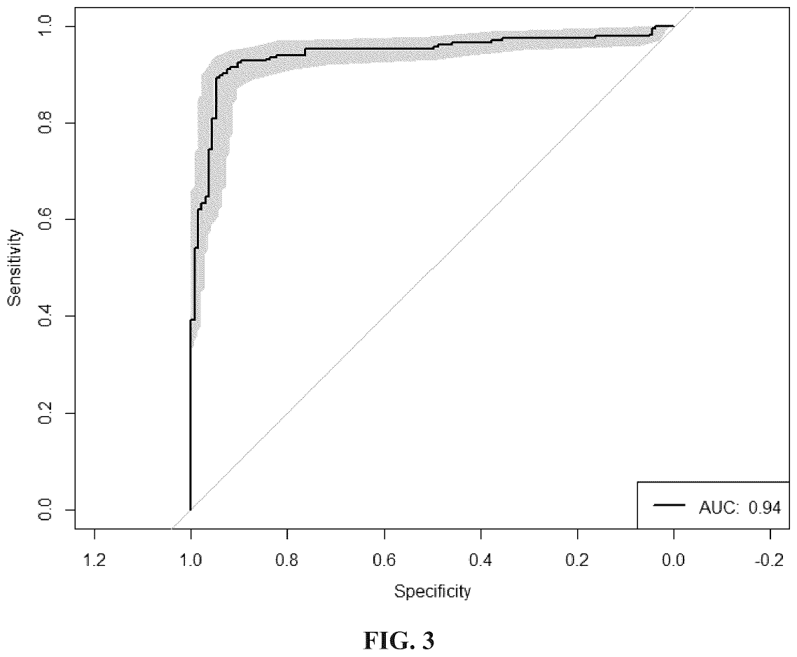

A quadratic discriminant analysis (QDA) classifier trained on the full retained miRNA panel provided the best overall binary classification performance in cross-validated training and test assessments. On the held-out test data the QDA model achieved: overall accuracy 91%, sensitivity (true positive rate) 0.89, and specificity (true negative rate) 0.92 (FIGS. 2A-F). The receiver operating characteristic (ROC) curve for the QDA model had an area under the curve (AUC) of 0.94 (FIG. 3), and bootstrap estimates of the 95% confidence interval for the ROC AUC confirmed that the model's discriminative ability was statistically robust on the test set. The confusion matrix for the QDA model indicates a low false-positive and false-negative rate consistent with the reported sensitivity and specificity.

### Discriminative Statistics and Calibration

In addition to ROC AUC, calibration analysis showed that the QDA model's probability outputs were well aligned with observed class frequencies across probability bins, supporting the model's utility for probabilistic decision-making. Bootstrap resampling of test folds produced narrow confidence intervals for accuracy and AUC, indicating stable performance given the available sample size and cross-validation scheme.

### Variable Importance and Minimal Panel Performance

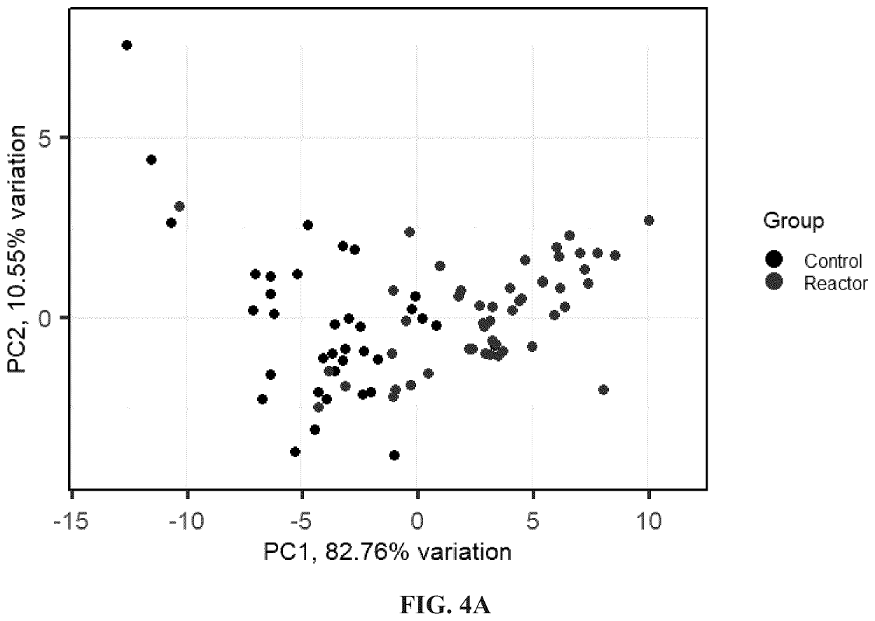

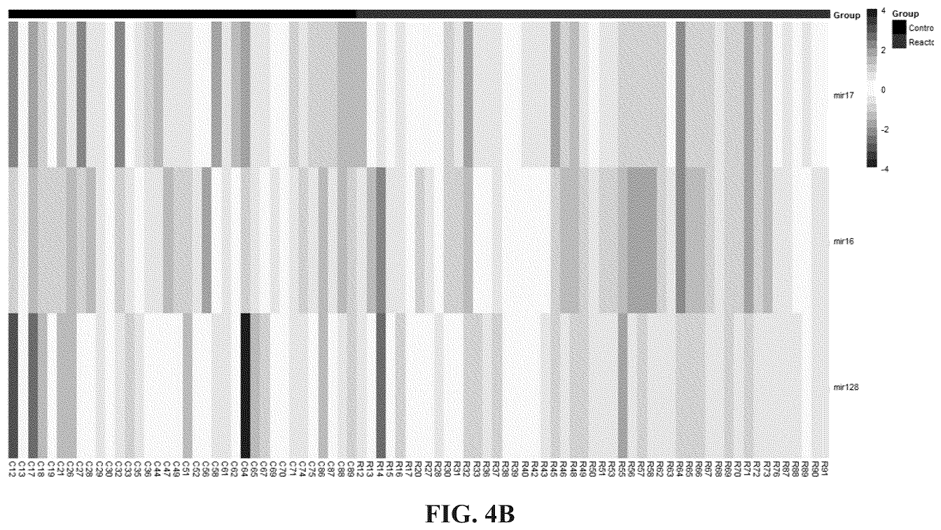

Variable importance analyses-including single-marker ROC analysis and model-specific importance measures-consistently identified miR-128, miR-17 and miR-16 as the most informative discriminators of reactor status when normalized to miR-21 and miR-423. Separation was evident both on PCA and heatmaps (FIGS. 4A and 4B, respectively).

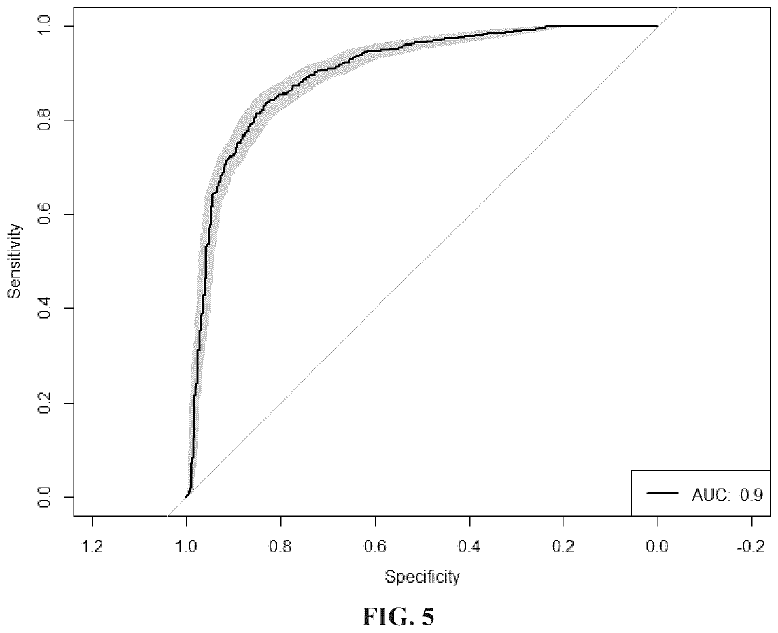

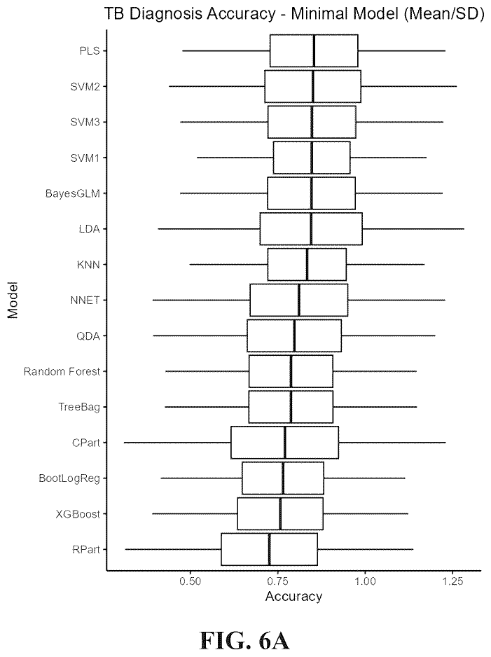

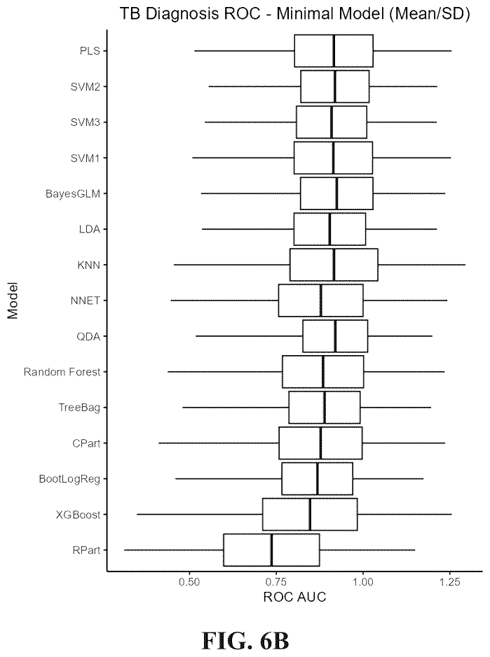

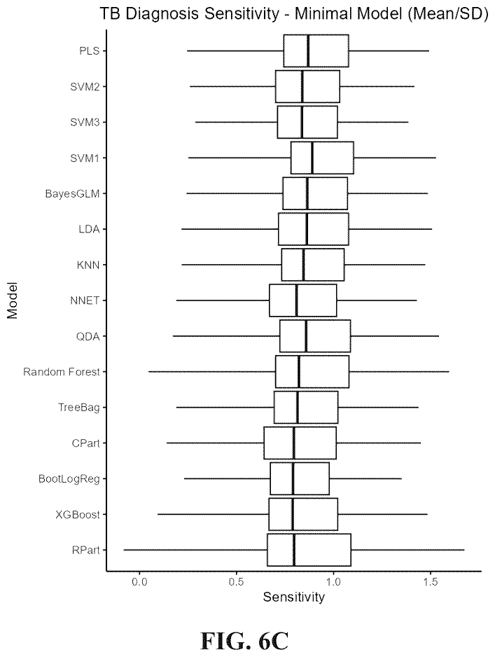

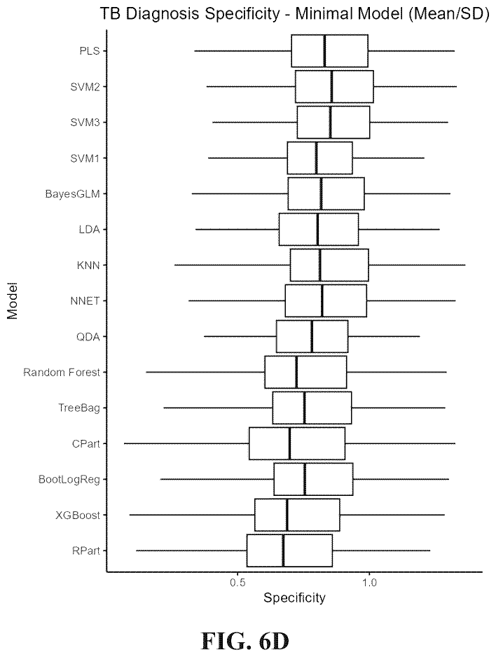

Using these ranked markers, a compact five-miRNA panel (the three informative markers plus the two normalizers) was evaluated with a partial least squares (PLS) classifier. The PLS minimal model achieved overall accuracy 87%, ROC AUC 0.89, sensitivity 0.87, and specificity 0.88 on the same test data (FIGS. 5 & 6A-F). These results indicate that a reduced marker set retains most of the discriminative power of the full panel while simplifying assay design and interpretation.

### Robustness and Practical Implications

Performance was estimated using repeated 10-fold cross-validation to reduce variance from fold assignment; reported metrics reflect aggregated out-of-sample performance. The combination of strong multivariate separation, high ROC AUC for the QDA model, and near-equivalent performance of the minimal PLS panel supports the feasibility of a qPCR-based miRNA diagnostic that is both accurate and amenable to a compact, lower-cost implementation. The minimal panel's slightly lower but comparable metrics suggest a practical trade-off between assay complexity and diagnostic performance suitable for field or near-patient deployment

### Results Summary

Bovine miRNA profiles measured by qPCR show clear group separation, with control and TB-reactor samples segregating along the first principal component and consistent differential patterns on hierarchical clustering heatmaps. A quadratic discriminant analysis (QDA) classifier trained on the retained miRNA panel achieved high discriminative performance on the test set (accuracy 91%, sensitivity 0.89, specificity 0.92; ROC AUC 0.94 with 95% confidence intervals indicating robust discrimination). Variable importance analyses identified miR-128, miR-17 and miR-16 (normalized to miR-21 and miR-423) as the most informative markers, and a compact five-miRNA partial least squares (PLS) model delivered near-equivalent performance (accuracy 87%, ROC AUC 0.89, sensitivity 0.87, specificity 0.88), supporting the feasibility of a reduced, qPCR-based diagnostic panel with rapid turnaround and strong classification accuracy.

### Example 2: Validation of Machine Learning Model

### Cohort and Sample Collection

A case-control cohort will be assembled for assay evaluation comprising control animals and TB reactors, controls as defined by negative SICCT skin testing and a negative TB farm history; positive cases defined by positive SICCT and identification of TB related lesions at post-mortem. Serum aliquots will be stored at between −20 and −80° C. until RNA extraction. All sample handling will follow standard biosafety and chain-of-custody procedures.

### RNA Extraction and Spike-In Control

Total RNA enriched for small RNAs will be extracted from a fixed volume of serum using a silica-column kit validated for miRNA recovery. A non-endogenous synthetic spike-in control (cel-miR-39) will be added to each sample prior to extraction at a fixed concentration to monitor extraction efficiency and technical variation. RNA yields and purity will be assessed by fluorometric quantification and inspection of extraction controls.

qPCR Assay and Data Acquisition

Reverse transcription and qPCR will be performed using TaqMan assays optimized for the panel of bovine miRNA. Each miRNA assay will be run in technical duplicate. Cq values will be recorded using a fixed fluorescence threshold and exported for downstream analysis. Assays with no amplification or Cq>35 will be flagged as undetermined and treated as missing data for that miRNA in that sample.

The quadratic discriminant analysis (QDA) classifier trained on either the full retained miRNA panel or the minimal five-miRNA panel (miR-128, miR-17 and miR-16 normalized to miR-21 and miR-423) as described above will be used to classify each sample as a control or reactor sample. The samples will be classified with an accuracy of about 91%.

### Example 3: Detection and Diagnosis of Bovine Tuberculosis

A blood, blood plasma, blood serum, urine, or saliva sample is taken from a cow with suspicion of bovine tuberculosis. RNA extraction and qPCR assay and data acquisition will be run as described above. The quadratic discriminant analysis (QDA) classifier trained on either the full retained miRNA panel or the minimal five-miRNA panel (miR-128, miR-17 and miR-16 normalized to miR-21 and miR-423) as described above will be used to classify the sample as a control or reactor sample. If the sample is a reactor sample, the cow will be diagnosed with bovine tuberculosis. In some embodiments wherein the sample is a reactor sample, the herd will be isolated. In some embodiments wherein the sample is a reactor sample, the cow will be isolated. In some embodiments wherein the sample is a reactor sample, the cow will be culled. In some embodiments wherein the sample is a reactor sample, the herd will be culled.

### Example 4: QPCR-Based miRNA Profiling with Machine Learning Optimization

### Materials and Methods

In this example, 16 candidate miRNAs (shown in Table 2) were selected based on reported differential expression, reproducibility across studies, and potential biological relevance to host immune response. These 16 miRNAs formed the initial assay panel for experimental evaluation.

TABLE 2

| SEQ ID | | |
|---|---|---|
| NO. | Name | Sequence |
| 2 | mir17 | CAAAGUGCUUACAGUGCAGGUAGU |
| 4 | mir16 | UAGCAGCACGUAAAUAUUGGUG |
| 6 | mir22 | AAGCUGCCAGUUGAAGAACUG |
| 7 | mir31 | AGGCAAGAUGCUGGCAUAGCU |
| 11 | mir150 | UCUCCCAACCCUUGUACCAGUGU |
| 14 | mir423 | UGAGGGGCAGAGAGCGAGACUUU |
| 15 | mir484 | UCAGGCUCAGUCCCCUCCCGAU |
| 16 | let7a | UGAGGUAGUAGGUUGUAUAGUU |
| 17 | let7b | UGAGGUAGUAGGUUGUGUGGUU |
| 22 | mir148a | UCAGUGCACUACAGAACUUUGU |
| 24 | mir320a | AAAAGCUGGGUUGAGAGGGCGA |
| 25 | mir320b | AGCUGGGUUGAGAGGGUGGU |
| 26 | mir374a | UUAUAAUACAACCUGAUAAGUG |
| 9 | mir128 | UCACAGUGAACCGGUCUCUUU |
| 8 | mir29a | CUAGCACCAUCUGAAAUCGGUUA |
| 3 | mir146a | UGAGAACUGAAUUCCAUAGGUUGU |

### Cohort and Sample Collection

A case-control cohort comprising 167 samples was assembled for assay evaluation: 70 control animals and 97 TB reactors: controls as defined by negative SICCT skin testing and a negative TB farm history; positive cases defined by positive SICCT and identification of TB related lesions at post-mortem or following experimental infection. Serum aliquots were stored at between −20 and −80° C. until RNA extraction. All sample handling followed standard biosafety and chain-of-custody procedures.

### RNA Extraction and Spike-In Control

Total RNA enriched for small RNAs was extracted from a fixed volume of serum using a silica-column kit validated for miRNA recovery. A non-endogenous synthetic spike-in control (cel-miR-39) was added to each sample prior to extraction at a fixed concentration to monitor extraction efficiency and technical variation. RNA yields and purity were assessed by fluorometric quantification and inspection of extraction controls.

qPCR Assay and Data Acquisition

Reverse transcription and qPCR were performed using TaqMan assays optimized for the panel of bovine miRNA. Each miRNA assay was run in technical duplicate. Cq values were recorded using a fixed fluorescence threshold and exported for downstream analysis. Assays with no amplification or Cq>35 were flagged as undetermined and treated as missing data for that miRNA in that sample.

### Normalization Strategy and Quality Control

miR-423 (SEQ ID NO: 14) was selected an endogenous normalizers because it exhibited the lowest variation across the cohort after initial normalization with NormFinder and geNorm (see Andersen, et al., “Normalization of real-time quantitative reverse transcription-PCR data: a model-based variance estimation approach to identify genes suited for normalization, applied to bladder and colon cancer data sets.”, Cancer Res. 64(15):5245-5250 (2004); Mestdagh, et al., “A novel and universal method for microRNA RT-qPCR data normalization”, Genome Biol. 10(6): R64 (2009), respectively). Samples with aberrant spike-in recovery or technical failure were excluded prior to downstream filtering.

### Data Filtering and Final Analysis Set

In order to obtain a complete data set, existing missing values (6.25% overall; particularly in mir193a, 19.76%, and mir106b, 18.56%) were imputed by estimated values based on the information from the observed miRNAs.

A cross-validation study seeking a balance between bias and variance in prediction was set up to evaluate the performance of a tailored collection of predictive models. The following results summarize the leading predictive performance provided by a random forest algorithm.

Model performance was assessed using the following metrics computed on held-out folds and aggregated across repeats: ROC AUC, overall accuracy, sensitivity (true positive rate), and specificity (true negative rate). Confidence intervals for ROC AUC were estimated using bootstrap resampling of test folds, a nonparametric resampling approach in which the observed test fold predictions are resampled with replacement to generate empirical distributions of ROC AUC values, from which percentile-based confidence intervals are calculated. The best performing model was identified by a combination of ROC AUC and balanced sensitivity/specificity.

### Results

### Classifier Performance—RF (Lead Model)

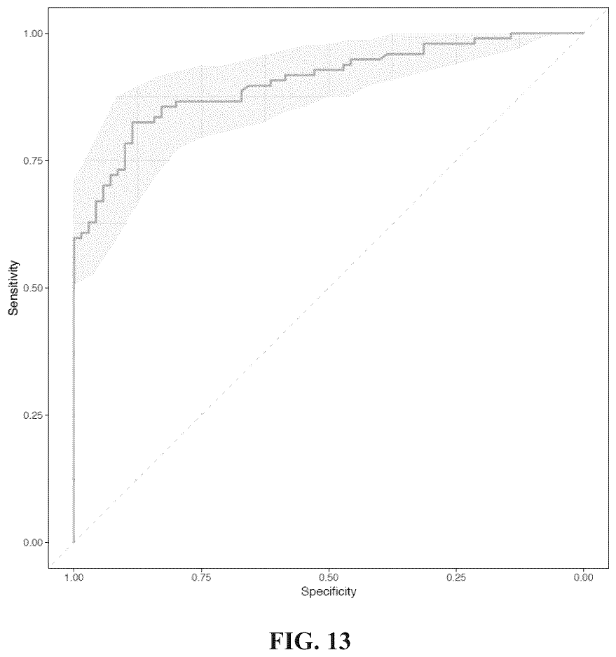

A random forest (RF) classifier trained on the full retained miRNA panel provided the best overall binary classification performance in cross-validated training and test assessments. On the held-out test data the RF model achieved: overall accuracy 84%, sensitivity (true positive rate) 0.85, and specificity (true negative rate) 0.93. The receiver operating characteristic (ROC) curve for the QDA model had an area under the curve (AUC) of 0.90 (FIG. 13), and bootstrap estimates of the 95% confidence interval for the ROC AUC confirmed that the model's discriminative ability was statistically robust on the test set.

### Example 5: QPCR-Based miRNA Profiling with Machine Learning Optimization with Mean Expression Normalization and Independent Standardization

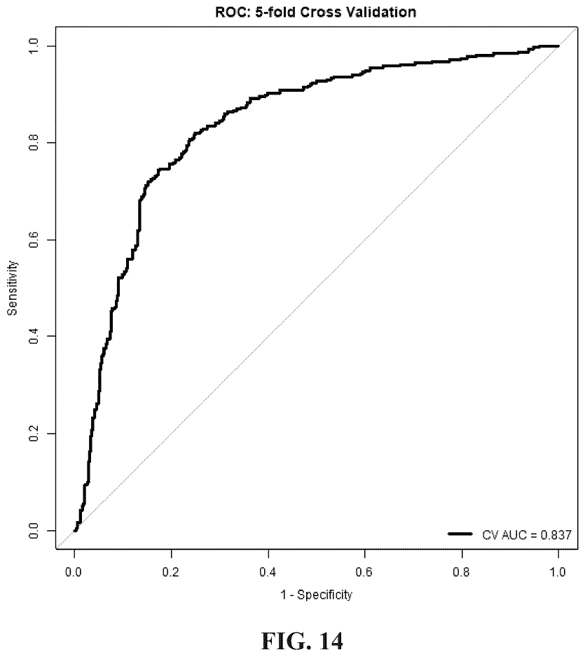

In this example, the unnormalized Cq data from Example 4 were analyzed using global mean normalization and independent standardization. As an alternative to imputing missing data, a greedy forward-selection heuristic was used to maximize data yield. Features were ranked by completeness and iteratively added to the panel to identify the largest complete matrix of samples and miRNA Cq values. These Cq values were treated directly as log-scale expression markers without conversion to relative quantities. For each sample, mean normalization was applied using techniques described by Willems et al (Willems, et al., “Standardization of real-time PCR gene expression data from independent biological replicates”, Anal Biochem. 379(1):127-129 (2008)) and Mestdagh et al. (Mestdagh, et al., “A novel and universal method for microRNA RT-qPCR data normalization.” 10(6):R64 (2009)) by calculating the arithmetic mean of the Cq values across all target miRNAs in that sample and subtracting this mean from each miRNAs Cq value to center values. Finally, independent standardization (Z-score scaling) was applied to the datasets separately to ensure all features had a mean of 0 and standard deviation of 1 within the batches. This methodology is not dependent on normalizing miRNA markers, providing an alternate method of analysis. Machine learning algorithm selection was performed as described in Example 1, leading to a support vector machines (SVM) model having the most robust metrics. In this example, an AUC of 0.837 was achieved across the average of the cross-validated folds, comparable to the normalizer dependent methodology (FIG. 14)

### Example 6: QPCR-Based miRNA Profiling with Machine Learning Optimization with Pair-Wise Scaling

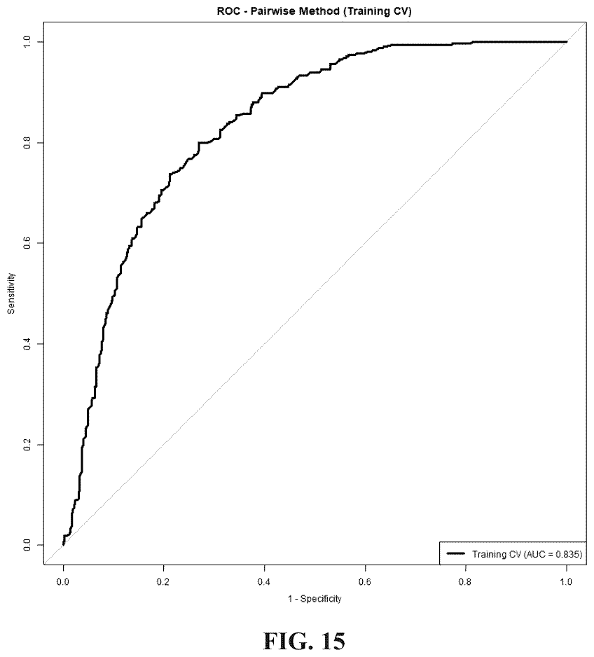

In this example, the largest complete submatrix was selected from Cq data from Example 4, as described in Example 5. Cq values were treated directly as log-scale expression markers without conversion to relative quantities. For each sample, pairwise normalization using a technique described in Geman et al (Geman, et al., “Classifying gene expression profiles from pairwise mRNA comparisons”, Statistical Applications in Genetics and Molecular Biology 3(1) (2004)) was applied by calculating the difference in Cq values between all possible pairs of target miRNAs. These differences represent relative expression ratios, creating self-normalizing features that capture the biological balance between markers without relying on a global mean or normalizing miRNA markers. Machine learning algorithm selection was performed as described in Example 1, leading to an SVM model having the most robust metrics. In this example, an AUC of 0.835 was achieved across the average of the cross-validated folds, comparable to the normalizer dependent and global mean normalization methodologies (FIG. 15).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A computer-implemented method for detecting Mycobacterium bovis infection in a subject, the method comprising:
receiving, by a processor, quantitative expression data for a panel of microRNA (miRNA) molecules from a biological sample obtained from the subject;
applying, by the processor, a trained classification model to the expression data, wherein the trained classification model is configured to classify the expression data as control or reactor; and
generating, by the processor, a classification output indicative of Mycobacterium bovis infection status of the subject based on the applying;
wherein the panel of miRNA molecules is selected from the group of miRNA molecules consisting of mir155, mir17, mir146a, mir16, mir1, mir22, mir31, mir29a, mir128, mir142, mir150, mir21, mir194, mir423, mir484, let7a, let7b, mir15b, mir99b, mir106b, mir146b, mir148a, mir193a, mir320a, mir320b, mir374a, mir21, and mir194.

2. The method of claim 1, wherein the panel of miRNA molecules comprises miR-128, miR-17, miR-16, miR-21, and miR-423.

3. The method of claim 1, further comprising a step of normalizing, by the processor, the quantitative expression data by:
a) calculating a delta quantification cycle (delta Cq) value for each target miRNA in the panel; and
b) normalizing the delta Cq values by one or more techniques selected from normalizing the delta Cq values relative to a mean quantification cycle (Cq) value of one or more endogenous control miRNAs, global mean normalization,
independent standardization, pairwise normalization, or a combination thereof;
wherein the processor then applies the trained classification model to the normalized expression data.

4. The method of claim 3, wherein the one or more endogenous control miRNAs comprise miR-21, miR-423, or a combination thereof, and the delta Cq value for each target miRNA is calculated relative to the mean Cq value of miR-21, miR-423, or a combination thereof.

5. The method of claim 1, wherein the trained classification model comprises either a classifier selected from: a quadratic discriminant analysis classifier, a partial least squares classifier, a random forest classifier, or a support vector machines (SMV) classifier; or an ensemble combination of two or more of a quadratic discriminant analysis classifier, a partial least squares classifier, a random forest classifier, or a support vector machines (SMV) classifier.

6. The method of claim 1, wherein the classification model generates a classification probability score indicative of a likelihood of Mycobacterium bovis infection status.

7. The method of claim 6, wherein the classification probability score is compared against a threshold value, and the classification output indicates control when the classification probability score is below the threshold and reactor when the classification probability score is at or above the threshold.

8. The method of claim 5, wherein the ensemble combination comprises averaging or combining classification probability scores from the two or more classifiers to produce a combined classification probability score.

9. The method of claim 1, wherein the trained classification model is trained on a reference dataset comprising expression profiles from control samples and reactor samples using cross-validated training.

10. The method of claim 9, wherein the cross-validated training comprises ten-fold cross-validation repeated five times.

11. The method of claim 1, wherein the biological sample is selected from the group consisting of serum, plasma, and whole blood.

12. A system for detecting Mycobacterium bovis infection, the system comprising:
a non-transitory memory storing a trained classification model configured to classify microRNA (miRNA) expression data as control or reactor, and instructions; and
a processor coupled to the non-transitory memory, wherein execution of the instructions by the processor causes the processor to:
receive quantitative expression data for a panel of miRNA molecules from a biological sample;
apply the trained classification model to the expression data; and
generate a classification output indicative of Mycobacterium bovis infection status;
wherein the panel of miRNA molecules is selected from the group of miRNA molecules consisting of mir155, mir17, mir146a, mir16, mir1, mir22, mir31, mir29a, mir128, mir142, mir150, mir21, mir194, mir423, mir484, let7a, let7b, mir15b, mir99b, mir106b, mir146b, mir148a, mir193a, mir320a, mir320b, mir374a, mir21, and mir194.

13. The system of claim 12, wherein execution of the instructions by the processor further causes the processor to: normalize the quantitative expression data by:
a) calculating a delta quantification cycle (delta Cq) value for each target miRNA in the panel; and
b) normalizing the delta Cq values by one or more techniques selected from normalizing the delta Cq values relative to a mean quantification cycle (Cq) value of one or more endogenous control miRNAs, global mean normalization,
independent standardization, pairwise normalization, or a combination thereof;
wherein the processor then applies the trained classification model to the normalized expression data.

14. The system of claim 12, wherein the panel of miRNA molecules comprises miR-17, miR-16, miR-22, miR-128, miR-148a, miR-320a, miR-320b, miR-21, miR-423, and miR-193a.

15. The system of claim 13, wherein the one or more endogenous control miRNAs comprise miR-21, miR-423, or a combination thereof, and the delta Cq value for each target miRNA is calculated relative to the mean Cq value of miR-21, miR-423, or a combination thereof.

16. The system of claim 12, wherein the trained classification model comprises either a classifier selected from: a quadratic discriminant analysis classifier, a partial least squares classifier, a random forest classifier, or a support vector machines (SMV) classifier; or an ensemble combination of two or more classifiers selected from: a quadratic discriminant analysis classifier, a partial least squares classifier, a random forest classifier, or a support vector machines (SMV) classifier.

17. The system of claim 12, wherein the trained classification model is trained on a reference dataset comprising expression profiles from control samples and reactor samples using cross-validated training.

18. The system of claim 17, wherein the cross-validated training comprises ten-fold cross-validation repeated five times.

19. The system of claim 12, wherein the panel of miRNA molecules comprises miR-128, miR-17, miR-16, miR-21, and miR-423.

20. The system of claim 12, wherein the system is configured to detect early infection or latent infection with Mycobacterium bovis.
